(19) United States
(12) Patent Application Publication
(10) Pub. No.: US 2021/0361716 A1
(43) Pub. Date: Nov. 25, 2021

(54) METHODS OF TREATING CEREBRAL PALSY AND HYPOXIC-ISCHEMIC ENCEPHALOPATHY USING HUMAN UMBILICAL CORD TISSUE-DERIVED MESENCHYMAL STROMAL CELLS

(71) Applicant: DUKE UNIVERSITY, Durham, NC (US)

(72) Inventors: Joanne Kurtzberg, Durham, NC (US); Jessica Sun, Durham, NC (US); Allen Song, Durham, NC (US); Charles Michael Cotten, Durham, NC (US)

(73) Assignee: DUKE UNIVERSITY, Durham, NC (US)

(21) Appl. No.: 17/044,744

(22) PCT Filed: Apr. 4, 2019
(86) PCT No.: PCT/US19/25796

§ 371 Date: Oct. 1, 2020

Related U.S. Application Data
(60) Provisional application No. 62/652,818, filed on Apr. 4, 2018.

(51) Int. Cl. A61K 35/28

(52) U.S. Cl. CPC A61K 35/28; A61K 9/0019

(57) ABSTRACT

The present invention relates to methods of treating cerebral palsy and hypoxic-ischemic encephalopathy using cord blood tissue-derived mesenchymal stromal cells.

### PRIORITY

This application claims the benefit of U.S. provisional Ser. No. 62/652,818, filed on Apr. 4, 2018, which is incorporated by reference herein in its entirety.

### STATEMENT REGARDING FEDERALLY SPONSORED RESEARCH OR DEVELOPMENT

This invention was made with government support under the Clinical and Translational Science Award (CTSA) Program, award number UL1TR002553, through the National Center for Advancing Translational Sciences (NCATS), National Institutes of Health. The United States government has certain rights in the invention.

### BACKGROUND OF THE INVENTION

### Field of the Invention

The present disclosure relates to methods of treating cerebral palsy and hypoxic-ischemic encephalopathy (HIE). More particularly, the present disclosure relates to methods of using cord blood tissue-derived mesenchymal stromal cells to treat cerebral palsy and HIE.

### Description of the Related Art

Children with cerebral palsy face a lifetime of disability, resulting in enormous physical, emotional, and financial burdens to affected patients, their parents, and society at large. Typically caused by an in utero or perinatal injury to the developing brain, cerebral palsy is the most common—and most costly—chronic motor disorder of childhood. The cornerstone of cerebral palsy treatment relies on countless hours of physical and occupational therapies that are entirely supportive. There is no treatment available to repair the brain damage that caused the disabilities. Thus, a novel therapy that could promote repair of damaged brain tissue has potential to reduce societal burden and to greatly improve survival, function, and quality of life for patients with cerebral palsy.

During the intrapartum period, compromised delivery of oxygen and blood flow to the fetal brain can lead to a significant brain injury that is clinically apparent in the first postnatal hours, described as hypoxic-ischemic encephalopathy (HIE). Moderate to severe neonatal hypoxic-ischemic encephalopathy (HIE) can lead to death or significant neurodevelopmental impairment (Kurinczuk et al., Early Hum Dev. 2010, 86(6):329-38; Lee et al., Pediatr Res. 2013, 74(Suppl 1): 50-72). Clinical trials of whole-body cooling demonstrated safety and efficacy, and therapeutic hypothermia (TH) has become standard for newborns with HIE (the NICHD trial: Shankaran et al., NEJM 2005, 353(15):1574-1584; the TOBY trial: Azzopardi et al., NEJM 2009, 361(14):1349-1358; the nEURO trial: Simbruner et al. Pediatrics 2010, 126(4):e771-778; the ICE trial: Jacobs et al., Archives of Pediatrics &Adolescent Medicine 2011, 165(8):692-700; NICHD workshop summary statement: Higgins et al., J Pediatr. 2011, 159(5):851-858). Despite hypothermia, one quarter to half of infants treated with hypothermia for moderate to severe encephalopathy either die or survive with neurologic impairment (Higgins et al., J Pediatr. 2011, 159(5):851-858; Shankaran et al., JAMA 2014, 312(24):2629-2639). The composite results of whole body hypothermia studies indicates a reduction in risk of death or impairment when hypothermia is initiated in the first 6 postnatal hours and continued for 72 hours; however, the effect is incompletely neuroprotective. In these studies, 44-51% of infants died or survived with disabilities, 24-38% of babies with HIE and were cooled died, and 13-28% of the survivors were later diagnosed with cerebral palsy (Shankaran et al., NEJM 2005, 353(15):1574-1584; Azzopardi et al., NEJM 2009, 361(14):1349-1358; Simbruner et al. Pediatrics 2010, 126(4):e771-778; Jacobs et al., Archives of Pediatrics &Adolescent Medicine 2011, 165(8):692-700). While cooling is helpful, the results of these trials provide strong incentive for development of adjunct therapies.

Mesencymal stromal cells (MSCs) are a heterogeneous group of undifferentiated, pluripotent cells that can be isolated from several different tissues including bone marrow, adipose tissue, and birth tissues (umbilical cord tissue, placenta). While MSCs can give rise to mesodermal tissue types including bone, cartilage, and fat, their primary mechanism of action is thought to result from immunomodulatory and other paracrine effects. MSCs have demonstrated a multitude of immunomodulatory effects on both humoral and cell-mediated immune responses. These include, but are not limited to, inhibiting B-, T-, NK, dendritic-cell, and microglial proliferation, decreasing pro-inflammatory cytokine production, and blocking neutrophil recruitment. In addition, numerous preclinical studies using MSC transplantation for diseases of the central nervous system suggest that MSCs can act through release of different neurotrophic, anti-inflammatory, and anti-apoptotic factors to promote recovery the injured area and prevent further damage (Dori et al., Histol Histopathol. 2017, 32(10):1041-1055; Mueller et al., Stem Cells Dev. 2017, 26(4):239-248; Pishiutta et al., Crit Care Med. 2016, 44(11):e1118-e1131; Xie et al., Med Sci Montt. 2016, 22:3552-3561; Cameron et al., Mol Cell Neurosci. 2015, 68:56-72). Despite their ability to modulate the immune response, MSCs themselves have low immunogenicity. MSCs express low levels of MHC class I molecules on their surface and lack the expression of MHC class II and several costimulatory molecules. This allows MSCs to be used in the allogeneic setting across HLA barriers, without the need for donor-recipient HLA matching. In fact, in a review of 13 human studies of intravenous allogeneic MSC administration, including 1,012 mostly adult patients, there were no reports of infusional toxicity (Lalu et al., PLoS One. 2012, 7(10):e47559), supporting the notion that MSCs are “immune-privileged” and can avoid immunological allorecognition. When utilized as a therapeutic cell, MSCs exert effects via trophic signaling. It is estimated that after infusion, MSCs survive in the recipient for up to 4 months. MSCs do not engraft in the recipient.

The present inventors and others have previously shown that umbilical cord blood (CB) and mesenchymal stromal cells (MSCs) lessen the clinical and radiographic impact of hypoxic brain injury and stroke in animal models. CB also engrafts and differentiates in brain, facilitating neural cell repair, in animal models and human patients with inborn errors of metabolism undergoing allogeneic, unrelated donor CB transplantation. The inventors believe that CB cells or human cord tissue-derived MSCs (hCT-MSC), acting primarily through paracrine mechanisms, could serve as vehicles for emerging cellular therapies in patients with brain injuries.

In prior studies, the inventors conducted safety studies and a phase II, randomized, double blind, placebo-controlled trial of autologous CB in children with cerebral palsy. In that study, children who were infused with ≥2×10⁷ cells/kg exhibited a greater degree of motor improvement than children who received lower doses or placebo. That study was limited by small sample size since many children with cerebral palsy do not have a banked autologous cord blood unit and by the inclusion of children 1-2 years of age for whom analysis of the predicted motor change score was not possible. The inventors also conducted a phase I safety study of sibling CB infusion in 15 patients with cerebral palsy, indicating that allogeneic partially HLA-matched CB infusion is safe in this patient population.

In neonatal animal models of HIE, infusions of human volume- and red-blood cell reduced nucleated cord blood cells (human umbilical cord blood (CB) cells) results in anatomic and functional improvement. Animal models of neonatal hypoxic-ischemic injury indicate that the mechanism appears to be paracrine, with increases in neurotrophic and anti-inflammatory factors in brain (Rosenkranz K and Meier C., Annals of Anat 2011, 193:371-379; Tsuji et al., Neuroscience 2014, 263:148-158; Drobyshevsky et al., Dev Neurosci. 2015, 37(4-5):349-62). In prior studies the inventors have reported outcomes of infants enrolled in a CTSI-supported phase I trial of autologous cells. In that study, and in a phase II study, cord blood is collected, red blood cell- and volume-reduced. In the phase II, two doses of cells or placebo are infused in the first 48 postnatal hours. In the phase I, fifty-one infants received cells. Two infants died (after hospital discharge). Twenty-five (64%) of 39 infants with known outcomes survived with one year developmental scores >85, which compares favorably with the hypothermia trials in which approximately 50% of cooled infants survived without moderate or severe impairment (Higgins et al., J Pediatr. 2011, 159(5):851-858). A challenge to generalizability of clinical trials, and potential future use of autologous cord blood cells for newborns with HIE has become apparent however: collecting cord blood cells at difficult deliveries. Collecting cord blood is not routine in most institutions. Having an ‘off-the-shelf,’ allogeneic cell-based product would allow for a readily available cellular intervention for newborn infants with moderate to severe HIE who did not have cord blood collected.

### SUMMARY OF THE INVENTION

The present invention offers a method of treating patients with cerebral palsy or HIE through the administration of allogeneic human umbilical cord tissue-derived mesenchymal stromal cells (hCT-MSCs). The present invention provides the benefit of eliminating the restriction of having an autologous CB unit.

In one aspect, the present invention comprises a method of treating a patient with cerebral palsy comprising administering a therapeutically effective amount of human allogeneic umbilical cord-derived mesenchymal stromal cells (hCT-MSCs) to the patient. In certain embodiments of this aspect of the invention, the hCT-MSCs are administered systemically. In certain embodiments, they are administered intravenously. In some embodiments of this aspect of the invention, the patient is administered hCT-MSCs three times in a six month period. In certain embodiments, the administration is at baseline, at three months, and at six months. In other embodiments of this aspect of the invention, the patient is administered hCT-MSCs at a dose of at least about 2×10⁶ cells/kg.

In a further aspect, the present invention comprises a method of treating a patient with HIE comprising administering a therapeutically effective amount of human allogeneic umbilical cord-derived mesenchymal stromal cells (hCT-MSCs) to the patient. In certain embodiments of this aspect of the invention, the hCT-MSCs are administered systemically. In certain embodiments, they are administered intravenously. In some embodiments of this aspect of the invention, the patient is administered hCT-MSCs three times in a six month period. In certain embodiments, the administration is at baseline, at three months, and at six months. In other embodiments of this aspect of the invention, the patient is administered hCT-MSCs at a dose of at least about 2×10⁶ cells/kg.

In yet a further aspect, the present invention comprises a method of treating a patient with HIE comprising administering a therapeutically effective amount of human allogeneic umbilical cord-derived mesenchymal stromal cells (hCT-MSCs) to the patient. In certain embodiments of this aspect of the invention, the patient with HIE is a newborn 36 weeks gestation or later, who suffers from moderate to severe hypoxic-ischemic neonatal encephalopathy. In certain embodiments of this aspect of the invention, the hCT-MSCs are administered systemically. In certain embodiments, they are administered intravenously. In some embodiments of this aspect of the invention, the patient is administered hCT-MSCs in a single dose in the first 48 postnatal hours. In some embodiments of this aspect of the invention, the patient is administered two doses of hCT-MSCs. In certain embodiments where the patient is administered two doses, the first dose is given in the first 48 postnatal hours, and the second dose is given approximately two months after the first dose. In other embodiments of this aspect of the invention, the patient is administered hCT-MSCs at a dose of at least about 2×10⁶ cells/kg. In certain embodiments of this aspect of the invention, the hCT-MSCs are administered in conjunction with therapeutic hypothermia.

### BRIEF DESCRIPTION OF THE DRAWINGS

FIG. 1 is a flow chart summarizing the Phase I/II trial to determine the effect size of change in GMFM-66 score in subjects treated with hCT-MSC compared to allogeneic CB.

FIG. 2 is a flow chart summarizing the Phase I Study of hCT-MSC, an Umbilical Cord-Derived Mesenchymal Stromal Cell Product, in newborn infants with moderate or severe hypoxic-ischemic neonatal encephalopathy.

### DETAILED DESCRIPTION OF THE INVENTION

Before the disclosed processes and materials are described, it is to be understood that the aspects described herein are not limited to specific embodiments, apparati, or configurations, and as such can, of course, vary. It is also to be understood that the terminology used herein is for the purpose of describing particular aspects only and, unless specifically defined herein, is not intended to be limiting.

It is also to be understood that unless clearly indicated otherwise by the context, embodiments disclosed for one aspect or embodiment of the invention can be used in other aspects or embodiments of the invention as well, and/or in combination with embodiments disclosed in the same or other aspects of the invention. Thus, the disclosure is intended to include, and the invention includes, such combinations, even where such combinations have not been explicitly delineated.

### Definitions

For the purposes of promoting an understanding of the principles of the present disclosure, reference will now be made to particular embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

“About” is used to provide flexibility to a numerical range endpoint by providing that a given value may be “slightly above” or “slightly below” the endpoint without affecting the desired result.

Recitation of ranges of values herein are merely intended to serve as a shorthand method of referring individually to each separate value falling within the range, unless otherwise indicated herein, and each separate value is incorporated into the specification as if it were individually recited herein. The recitation is also intended to refer individually to each sub-range falling within the broader range recited, and each separate sub-range is incorporated into the specification as if it were individually recited herein. For example, if a range is stated as 1% to 50%, it is intended that values such as 2% to 40%, 10% to 30%, or 1% to 3%, etc., and values such as 2%, 10%, 30%, 40%, and 50%, etc. are expressly enumerated in this specification. These are only examples of what is specifically intended, and all possible numbers, and combinations of numerical values between and including the lowest value and the highest value enumerated are to be considered to be expressly stated in this disclosure.

As used herein, “treatment,” “therapy,” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. In some embodiments, the disease comprises cerebral palsy. In other embodiments, the disease comprises hypoxic-ischemic encephalopathy.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. Preferably, the subject is a human patient that has, or is suffering from, cerebral palsy or a hypoxic-ischemic brain injury.

As used herein, the term “disease” refers to any condition that is abnormal, such as a disorder or a structure or function that affects part or all of a subject. In some embodiments, the disease comprises a neurological disorder. In certain embodiments, the neurological disorder comprises cerebral palsy; in other embodiments, the neurological disorder comprises a hypoxic-ischemic brain injury.

As used herein, the term “cerebral palsy” (CP) refers to any one of a number of neurological disorders that appear in infancy or early childhood and permanently affect body movement and muscle coordination but don't worsen over time. While cerebral palsy affects muscle movement, it isn't caused by problems in the muscles or nerves, but rather by abnormalities in parts of the brain that control muscle movements. The majority of children with cerebral palsy are born with it, or develop it as a result of a brain injury associated with the birthing process or in the neonatal period (e.g. neonatal hypoxic-ischemic encephalopathy), although it may not be detected until months or years later. The early signs of cerebral palsy usually appear before a child reaches 3 years of age. The most common are a lack of muscle coordination when performing voluntary movements (ataxia); stiff or tight muscles and exaggerated reflexes (spasticity); walking with one foot or leg dragging; walking on the toes, a crouched gait, or a “scissored” gait; and muscle tone that is either too stiff or too floppy.

As used herein, the term “hypoxic-ischemic encephalopathy” (HIE) refers to the brain injury that results from compromised delivery of oxygen and blood flow to the fetal brain during the intrapartum period. Moderate to severe neonatal HIE can lead to death or significant neurodevelopmental impairment.

Unless otherwise defined, all technical terms used herein have the same meaning as commonly understood by one of ordinary skill in the art to which this disclosure belongs.

Treatment of Cerebral Palsy or Hypoxic-Ischemic Encephalopathy

In view of the present disclosure, the methods described herein can be configured by the person of ordinary skill in the art to meet the desired need. In general, the disclosed materials, methods, and apparati provide methods of treating a subject having cerebral palsy or HIE comprising, consisting of, or consisting essentially of administering to the subject a therapeutically effective amount of hCT-MSCs and/or a component or mixture of components thereof, such that the cerebral palsy or HIE is treated.

It is to be understood that as used herein, unless stated otherwise, the term “hCT-MSC” is meant to encompass any format and/or a component or mixture of components thereof, whether specifically so stated or not.

The patient may be any human or nonhuman animal. In one embodiment, the patient is human. In another embodiment, the patient is a human child under 18 years of age, or in any age range falling within this broader age range. In non-limiting examples, the patient may be a newborn, an infant 1-12 months old, 1 month to 2 years old, 1 year to 18 years old, 1 year to 17 years old, 1 year to 16 years old, 1 year to 15 years old, 1 year to 14 years old, 1 year to 13 years old, 1 year to 12 years old, 1 year to 11 years old, 1 year to 10 years old, 1 year to 9 years old, 1 year to 8 years old, 1 year to 7 years old, 1 year to 6 years old, 1 year to 5 years old, 1 year to 4 years old, 1 year to 3 years old, 1 year to 2 years old, 2 years to 18 years old, 2 years to 17 years old, 2 years to 16 years old, 2 years to 15 years old, 2 years to 14 years old, 2 years to 13 years old, 2 years to 12 years old, 2 years to 11 years old, 2 years to 10 years old, 2 years to 9 years old, 2 years to 8 years old, 2 years to 7 years old, 2 years to 6 years old, 2 years to 5 years old, 2 years to 4 years old, 2 years to 3 years old, 3 years to 18 years old, 3 years to 17 years old, 3 years to 16 years old, 3 years to 15 years old, 3 years to 14 years old, 3 years to 13 years old, 3 years to 12 years old, 3 years to 11 years old, 3 years to 10 years old, 3 years to 9 years old, 3 years to 8 years old, 3 years to 7 years old, 3 years to 6 years old, 3 years to 5 years old, 3 years to 4 years old, 4 years to 18 years old, 4 years to 17 years old, 4 years to 16 years old, 4 years to 15 years old, 4 years to 14 years old, 4 years to 13 years old, 4 years to 12 years old, 4 years to 11 years old, 4 years to 10 years old, 4 years to 9 years old, 4 years to 8 years old, 4 years to 7 years old, 4 years to 6 years old, 4 years to 5 years old, 5 years to 18 years old, 5 years to 17 years old, 5 years to 16 years old, 5 years to 15 years old, 5 years to 14 years old, 5 years to 13 years old, 5 years to 12 years old, 5 years to 11 years old, 5 years to 10 years old, 5 years to 9 years old, 5 years to 8 years old, 5 years to 7 years old, 5 years to 6 years old, 6 years to 18 years old, 6 years to 17 years old, 6 years to 16 years old, 6 years to 15 years old, 6 years to 14 years old, 6 years to 13 years old, 6 years to 12 years old, 6 years to 11 years old, 6 years to 10 years old, 6 years to 9 years old, 6 years to 8 years old, 6 years to 7 years old, 7 years to 18 years old, 7 years to 17 years old, 7 years to 16 years old, 7 years to 15 years old, 7 years to 14 years old, 7 years to 13 years old, 7 years to 12 years old, 7 years to 11 years old, 7 years to 10 years old, 7 years to 9 years old, or 7 years to 8 years old.

In some embodiments, the patient is a human up to about 45 years of age, or in any age range falling within the broader age range from about 1 year old to about 45 years old. For example, about 18 to about 45 years old, about 20 to about 45 years old, about 25 to about 45 years old, about 30 to about 45 years old, about 35 to about 45 years old, or about 40 to about 45 years old. In certain embodiments, the patient is a human of any age between 1 and 45 years old. For example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 years old.

In some embodiments, the patient is a human up to about 90 years of age, or any age range falling within the broader age range from about 1 year old to about 90 years old. For example, about 18 to about 90 years old, about 20 to about 90 years old, about 45 to about 90 years old, or about 60 to about 90 years old. In certain embodiments, the patient is a human of any age having a value between 45 and 90, even if not specifically enumerated herein, for example 45, 50, 55, 60, 65, 70, 75, 80, 85, or 90.

In certain embodiments, the patient is a newborn 36 weeks gestation or later, 37 weeks gestation or later, 38 weeks gestation or later, 39 weeks gestation or later, or 40 weeks gestation or later.

The hCT-MSCs may be administered to a subject by any technique known in the art, including local or systemic delivery. Routes of administration include, but are not limited to, subcutaneous, intracutaneous, intramuscular, intraperitoneal, intravenous, intrathecal, intracerebral, intraventricular, or epidural injection or implantation; topical administration; intratracheal; and intranasal administration. The hCT-MSCs may be administered by infusion techniques. Typically, infusion means that the hCT-MSCs are administered intravenously or subcutaneously. In some embodiments, the hCT-MSCs are administered systemically. In further embodiments, the hCT-MSCs are administered by intravenous injection.

Preparation of hCT-MSCs

The human allogeneic umbilical cord-derived mesenchymal stromal cells may be prepared, preserved, and prepared for administration by any methods known in the art. In some instances, the hCT-MSCs may be prepared in a clean room by cutting cord tissue into pieces and mincing and digesting with hyaluronidase, DNase, collagenase, and papain. The resultant cell suspension may then be plated in culture, grown to confluence to establish the P0 culture, and cryopreserved. P1 and P2 cultures may be grown under similar conditions and removed from cultureware. The final product may be derived from the P2 cultures which are harvested into plasmalyte with 5% human serum albumin, washed and cryopreserved in compartment cryobags containing 50-100 million cells in a final concentration of 10% DMSO with dextran. On the day of administration, one compartment may be thawed, diluted in 10-40 mLs of plasmalyte IV solution, placed in a syringe or bag and transported to the bedside for administration.

Administration of hCT-MSCs

The route of administration of the cord blood may be selected by one of skill in the art based on the diseases treated and desired results. Thus, in certain embodiments, the hCT-MSCs are administered via peripheral intravenous (IV) infusion.

In some embodiments, the hCT-MSCs may be administered as a single dose. In certain embodiments, the hCT-MSCs may be administered in multiple doses (e.g., two, three, or four or more single doses per treatment) over a time period (e.g., days or months). In some instances, the patient may be administered hCT-MSCs three times, and in some instances, three times in a six month period. In certain instances, the administration may be at baseline, at three months, and at six months. In certain instances, administration may be at baseline, at two months, and at four months. One of skill in the art will be able to derive variances in the dosing protocol as provided herein and understand that such variances are encompassed by the present invention.

In some instances, for a newborn patient with HIE, the patient may be administered hCT-MSCs once, or the patient may be administered hCT-MSCs twice. In some instances, the newborn may be administered hCT-MSCs thrice or more. In certain instances, a first dose is administered in the first 48 postnatal hours. In certain instances where a second dose is administered, the first dose is given in the first 48 postnatal hours, and the second dose is given approximately two months after the first dose. One of skill in the art will be able to derive variances in the dosing protocol as provided herein and understand that such variances are encompassed by the present invention.

Prior to administration of the hCT-MSCs, the patient may be premedicated as appropriate with, e.g. an antihistamine and/or a steroid.

When administered intraveneously, the hCT-MSCs may be administered over a period of time ranging from 20 minutes to about 75 minutes, e.g., over about 20 minutes to about 60 minutes, or over about 20 minutes to about 50 minutes, or over about 20 minutes to about 40 minutes, or over about 20 minutes to about 30 minutes, or over about 25 minutes to about 70 minutes, or over about 25 minutes to about 60 minutes, or over about 25 minutes to about 50 minutes, or over about 25 minutes to about 40 minutes, or over about 30 minutes to about 70 minutes, or over about 30 minutes to about 60 minutes, or over about 30 minutes to about 50 minutes, or over about 30 minutes to about 40 minutes. In some embodiments, the dose is administered over 30 minutes.

One of skill in the art will be able to derive a suitable dosing regimen for the invention. In some embodiments, a therapeutically effective dose of hCT-MSCs comprises a dose of about 1×10⁶ to about 6×10⁶ cells/kg patient weight, e.g., about 1×10⁶ to about 3×10⁶ cells/kg. about 1×10⁶ to about 2.5×10⁶ cells/kg, about 1.5×10⁶ to about 3×10⁶ cells/kg, or about 1.5×10⁶ to about 2.5×10⁶ cells/kg. In some instances, the hCT-MSCs may be administered at a dose of at least 2×10⁶ cells/kg patient weight.

One of skill in the art will recognize that suitable volume of the dose may be selected based on the desired route of administration. For example, intravenous administration may use dose volumes in the range of about 5 mL to about 50 mL; e.g., about 5 mL to about 40 mL, or about 5 mL to about 30 mL, or about 5 mL to about 20 mL, or about 5 mL to about 15 mL, or about 10 mL to about 40 mL, or about 10 mL to about 30 mL, or about 10 mL to about 20 mL, or about 10 mL to about 15 mL, or about 20 mL to about 50 mL, or about 20 mL to about 40 mL, or about 20 mL to about 30 mL, or about 30 mL to about 50 mL, or about 30 mL to about 40 mL, or about 40 mL to about 50 mL.

In certain embodiments, the hCT-MSCs are administered by infusion of 3 doses of 2×10⁶ cells/kg body weight, each infusion given three months apart, e.g. a first dose at baseline, a second dose at 3 months, and a 3 dose at 6 months. In other embodiments, the hCT-MSCs are administered by infusion of 2 doses of 2×10⁶ cells/kg body weight, one at birth (i.e. in the first 48 postnatal hours) and a second dose at 2 months.

Where peripheral IV administration is used, IV fluids may be administered at about 1.0 to about 2.0 times maintenance. For example, IV fluids may be administered post-infusion at about or 1.0 to about 1.5 times maintenance, or about 1.5 to about 2.0 times maintenance. The maintenance IV fluids may be administered for about 30 minutes to about 60 minutes after the infusion of hCT-MSCs. For example, maintenance IV fluids may be administered post-infusion for, e.g., about 30 minutes to about 60 minutes, or about 30 minutes to about 45 minutes, or about 45 minutes to about 60 minutes.

Any suitable intravenous fluids may be used for maintenance post infusion of hCT-MSCs. In certain embodiments, the maintenance IV fluid is a saline solution or Ringer's lactate solution. In certain embodiments, the maintenance IV fluid is 0.25% normal saline solution. In certain embodiments, the maintenance IV fluid is 0.5% normal saline solution.

In certain embodiments, where the administration of hCT-MSCs is in a patient with HIE, the hCT-MSCs may be administered in conjunction with therapeutic hypothermia. By “in conjunction with” it is meant prior to, at the same time/during, or after therapeutic hypothermia. In certain embodiments, the hCT-MSCs are administered at the same time as/during therapeutic hypothermia.

Certain aspects of the disclosure are now explained further via the following non-limiting examples.

### EXAMPLES

### Example 1: Phase I/II Trial to Determine the Effect Size of Change in GMFM-66 Score in Subjects Treated with hCT-MSC Compared to Allogeneic CB

Overview

This study is a phase I/II, prospective, randomized, open-label trial designed to determine the effect size of change in GMFM-66 score in subjects treated with hCT-MSC or allogeneic CB and assess the safety of repeated doses of hCT-MSC in children with cerebral palsy. Children ages 2-5 years with cerebral palsy due to hypoxic ischemic encephalopathy, stroke, or periventricular leukomalacia may be eligible to participate. All participants will ultimately be treated with an allogeneic cell product at some point during the study. Participants will be randomized to one of three arms: (1) the “AlloCB” arm will receive one allogeneic CB infusion at the baseline visit; (2) the “MSC” arm will receive three hCT-MSC infusions, one each at baseline, three months, and six months; (3) the “natural history” arm will not receive an infusion at baseline but will receive an allogeneic CB infusion at 12 months. Motor outcome measures will be assessed at baseline, six-months, and one-year time points. Safety will be evaluated at each infusion visit and remotely for an additional 12 months after the final visit. Duration of study participation will be 24 months from the time of baseline visit. Randomization to treatment arms will be stratified by age and GMFCS level at study entry.

The primary endpoint is the difference between a participant's observed and expected changes in GMFM-66 score 12 months after the initial study infusion. Interval estimates will be reported separately for the hCT-MSC, AlloCB, and Natural History arms. Expected GMFM-66 scores at 12 months will be calculated based on the participant's baseline age, GMFCS level, and GMFM-66 score at study entry using published reference percentiles (Hanna et al., Phys Ther. 2008, 88(5):596-607).

Purpose

The main purpose of this study is to estimate change in motor function 12 months after treatment with a single dose of allogeneic umbilical cord blood (AlloCB) or repeated doses of umbilical cord tissue-derived mesenchymal stromal cells (hCT-MSC) in children with cerebral palsy. In addition, this study will contribute much needed data to the clinical trials community on the natural history of the motor function in CP over shortterm (less than 1 year) time periods relevant to the conduct of clinical trials and assess the safety of AlloCB and hCT-MSC infusion in children with cerebral palsy.

Source of Unrelated CB Units for this Trial

The Carolinas Cord Blood Bank (CCBB) is one of the largest public cord blood banks in the nation. Established in 1998 with support from the National Heart and Blood Institute of the NIH, the CCBB has over 30,000 CB units in inventory and has distributed over 2,500 CB units for transplant to date. In 2012 the CCBB received approval from the FDA for its BLA application to market DUCORD, a stem cell product derived from umbilical cord blood, for use in transplants between unrelated donors and recipients. DUCORD is approved for use in hematopoietic stem cell reconstitution for patients with disorders affecting the hematopoietic system that are inherited, acquired, or result from myeloablative treatment. The CCBB currently collects from 10 hospital sites (8 in North Carolina, 1 in Atlanta, Ga. and 1 in Boston, Mass.). It also accepts CB donations from mothers delivering in any hospital in North Carolina and Atlanta through a kit donation program.

Specifications for Qualification CB Units

Based on established criteria utilizing allogeneic CB for hematopoietic stem cell transplantation and our experience in treating more than 600 children with autologous CB for neurological conditions, we have established the following criteria to qualify banked CB units for cell therapy studies. All CB units utilized for this current study will be obtained from the Carolinas Cord Blood Bank. The CB unit must have:
1. Pre-cryopreservation total nucleated cell count (TNCC) documented and at least 12×10⁷/kg
2. Pre-cryopreservation viability ≥85% of total cells and ≥70% of CD34+ cells
3. Pre-cryopreservation sterility culture performed and negative
4. Maternal infectious disease screening as follows: Testing must include negative results for Hepatitis B, Hepatitis C, HIV, HTLV, and syphilis. Additional screening, which is dependent on the timing of the CB collection, may be performed based on local and national regulations. Units from mothers who have a positive CMV antibody screen may be used.
5. Test sample available for identity confirmation and potency testing
6. HLA typing performed and meets study-specific parameters
7. CD45+ viability ≥40% and CD34+ viability ≥70% on thawed test sample

Source of MSCs for this Study: hCT-MSC

hCT-MSC is a third party MSC product manufactured from allogeneic donor digested umbilical cord tissue that is expanded for two passages in culture, cryopreserved, stored in the vapor phase of liquid nitrogen, and banked. The umbilical cord tissue is donated by healthy mothers delivering healthy full term babies after a normal pregnancy with written informed consent. The cells are manufactured, cryopreserved and stored in the Robertson CT2 GMP laboratory (Duke University, Durham, N.C.).

Umbilical cord tissue is an attractive source of MSCs as it is readily available and easily obtained without consequence to the donor, is non-controversial, and has a higher proliferative potential than MSCs from other postnatal sources (Drela et al., Cytotherapy. 2016, 18(4):497-509). Numerous preclinical studies have not demonstrated any evidence of tumorigenicity or toxicity of cord tissue derived MSCs (Park et al. Toxicol Res. 2016, 32(3):251-258). In early phase clinical trials published in English that utilized cord tissue-derived MSCs, in these 36 studies, including 695 patients and at least 1,416 doses of cord tissue-derived MSCs with follow up ranging from three months up to six years, no severe adverse events were reported. Several more clinical trials of cord-tissue derived MSCs in various disease conditions are underway (clinicaltrials.gov).

Study Rationale and Hypotheses

Previous studies suggest that adequately dosed autologous CB infusion can improve motor function in children with cerebral palsy. As it is not feasible that every child with cerebral palsy will have access to their autologous CB, this study will assess efficacy of two allogeneic sources of cells that can be available to all patients in need. The major goal of this study is to investigate change in motor function 12 months after treatment with two allogeneic cell sources, allogeneic CB and hCT-MSCs.

This study will generate important data regarding the effect size of change in motor function of these two cell sources and a natural history cohort to aid in the planning of future trials. The rationale for the study and for the potential benefit of cell therapy in cerebral palsy is based upon the following hypotheses:
We have demonstrated safety and dose-dependent efficacy of autologous CB infusions in children with cerebral palsy.
It is possible that different cell types, e.g. cord blood mononuclear cells versus cord tissue MSCs, may influence brain connectivity by different mechanisms.
Multiple doses of cells may be superior to a single dose of cells.
The developing brain exhibits remarkable plasticity, making young children ideal candidates for deriving maximal therapeutic benefit from restorative therapies, including CB.
CB cells, acting through paracrine mechanisms, may facilitate endogenous repair mechanisms and promote formation of new neural connections the motor cortex resulting in significant clinical improvements.
Brain connectivity plays an important role in the pathophysiology, and potentially mechanism of repair, of brain injury in children with cerebral palsy. Specifically, we hypothesize that (1) impairments in brain connectivity account for the motor deficits in children with cerebral palsy, (2) increases in brain connectivity have a direct impact on functional improvements, (3) children with cerebral palsy who receive CB infusions will exhibit greater increases in brain connectivity than children who receive placebo infusions, and (4) the severity of baseline brain connectivity abnormalities predict the potential for benefit of CB therapy.

Study Design

This study is a phase I/II, prospective, randomized, open-label trial designed to assess the effect size of change in GMFM-66 score in subjects treated with hCT-MSC or allogeneic CB and assess the safety of repeated doses of hCT-MSC in young children with cerebral palsy. Children ages 2-5 years with cerebral palsy due to hypoxic ischemic encephalopathy, stroke, or periventricular leukomalacia may be eligible to participate. All participants will ultimately be treated with an allogeneic cell product at some point during the study. Participants will be randomized to one of three arms: (1) the “AlloCB” arm will receive one allogeneic CB infusion at the baseline visit; (2) the “MSC” arm will receive three hCT-MSC infusions, one each at baseline, three months, and six months; (3) the “natural history” arm will not receive an infusion at baseline but will receive an allogeneic CB infusion at 12 months. All participants will have an initial clinical evaluation to verify and classify the diagnosis of cerebral palsy and determine eligibility. They will return for study visits an additional two (AlloCB and natural history arms) or three (MSC arm) times. Outcome measures will be assessed at baseline, six-months, and one-year time points. Additional safety endpoints will be assessed remotely for 12 months after the final in-person visit.

Study Objectives

Primary Objective: To determine the effect size of change in GMFM-66 score in children with cerebral palsy treated with a single dose of 10×10⁷ cells/kg of allogeneic CB or three doses of 2×10⁶ cells/kg of hCT-MSC.

Secondary Objective: To assess the safety of repeated doses of hCT-MSC in children with cerebral palsy.

Exploratory Objectives: (1) To determine the change in the Peabody Developmental Motor Scale-2 (PDMS-2) score at 6 and 12 months in children treated with allogeneic CB or hCT-MSC. (2) To analyze the change in normalized total brain connectivity, as measured by brain MRI with DTI, from baseline to 12 months. (3) To assess changes functional and quality of life measures at 6 and 12 months.

Study Design—General Design

This study is a phase I/II, prospective, randomized, open-label trial designed to determine the effect size of change in GMFM-66 score in subjects treated with hCT-MSC or allogeneic CB and assess the safety of repeated doses of hCT-MSC in children with cerebral palsy. Children ages 2-5 years with cerebral palsy due to hypoxic ischemic encephalopathy, stroke, or periventricular leukomalacia may be eligible to participate.

All participants will ultimately be treated with an allogeneic cell product at some point during the study. Participants will be randomized to one of three arms: (1) the “AlloCB” arm will receive one allogeneic CB infusion at the baseline visit; (2) the “MSC” arm will receive three hCT-MSC infusions, one each at baseline, three months, and six months; (3) the “natural history” arm will not receive an infusion at baseline but will receive an allogeneic CB infusion at 12 months. Motor outcome measures will be assessed at baseline, six-months, and one-year time points. Safety will be evaluated at each infusion visit and remotely for an additional 12 months after the final visit. Duration of study participation will be 24 months from the time of baseline visit. Randomization to treatment arms will be stratified by age and GMFCS level at study entry.

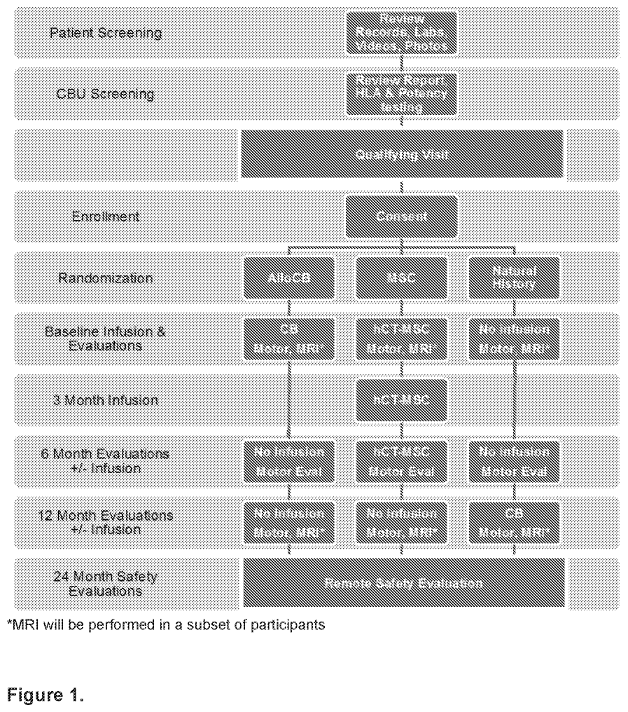

A study flow chart is provided in FIG. 1.

Study Design—Study Endpoints

Primary Endpoint: The primary endpoint is the difference between a participant's observed and expected changes in GMFM-66 score 12 months after the initial study infusion. Interval estimates will be reported separately for the hCT-MSC, AlloCB, and Natural History arms. Expected GMFM-66 scores at 12 months will be calculated based on the participant's baseline age, GMFCS level, and GMFM-66 score at study entry using published reference percentiles (Hanna et al. Phys Ther. 2008, 88(5):596-607).

Secondary Endpoints: The secondary endpoint is the number of adverse events occurring over the 12-month period post-infusion with hCT-MSC or AlloCB.

Exploratory Analyses:
Observed GMFM-66 score at baseline, 6, and 12 months
Change in the Peabody Developmental Motor Scale-2 (PDMS-2) score at 6 and 12 months.
Change in normalized total brain connectivity, as measured by brain MRI with DTI, from baseline to 12 months.
Change in functional and quality of life measures at 6 and 12 months.

Research Participant Selection and Withdrawal—Study Population

Ninety children ages 2-5 years with spastic cerebral palsy.

Research Participant Selection and Withdrawal—Inclusion Criteria
1. Age ≥24 months and ≤60 months adjusted age at the time of enrollment. Patient age
will be adjusted for prematurity if the patient was born at <37 weeks gestation.
2. Diagnosis: Unilateral or bilateral spastic cerebral palsy secondary to in utero or perinatal stroke/hemorrhage, hypoxic ischemic encephalopathy (including, but not limited to, birth asphyxia), and/or periventricular leukomalacia.
3. Performance status: Gross Motor Function Classification Score levels I-IV
4. Review of brain imaging (obtained as standard of care prior to study entry) does not suggest a genetic condition or brain malformation.
5. Legal authorized representative consent.

Research Participant Selection and Withdrawal—Exclusion Criteria
1. Available qualified autologous cord blood unit.
2. Hypotonic or ataxic cerebral palsy without spasticity.
3. Autism and autistic spectrum disorders without motor disability.
4. Hypsarrhythmia.
5. Intractable seizures causing epileptic encephalopathy.
6. Evidence of a progressive neurologic disease.
7. Has an active, uncontrolled systemic infection or documentation of HIV+ status.
8. Known genetic disease or phenotypic evidence of a genetic disease on physical exam.
9. Concurrent genetic or acquired disease or comorbidity(ies) that could require a future allogeneic stem cell transplant.
10. Requires ventilatory support, including home ventilator, CPAP, BiPAP, or supplemental oxygen.
11. Impaired renal or liver function as determined by serum creatinine >1.5 mg/dL and/or total bilirubin >1.3 mg/dL except in patients with known Gilbert's disease.
12. Possible immunosuppression, defined as WBC <3,000 cells/mL or absolute lymphocyte count (ALC)<1500 with abnormal T-cell subsets.
13. Patient's medical condition does not permit safe travel.
14. Previously received any form of cellular therapy.

Research Participant Selection and Withdrawal—Research Participant Recruitment and Screening

Patients may be recruited through IRB-approved advertising for the study on the websites of CB banks, parent sponsored websites, the NMDP website, selected cerebral palsy societies, local medical providers, and through a record of inquiries for previous studies (brain injury database. Separate IRB approval will be obtained for any advertisements.

Screening for this study is conducted under a separate, IRB-approved screening protocol (Pro00063563). Under this protocol, after written informed consent is obtained from a parent/guardian, the patient's medical records, videos, and results of brain imaging are obtained and reviewed. The medical review is conducted by a team of pediatric nurses, nurse practitioners, and physicians to identify the presence of any exclusion criteria. If no exclusion criteria are identified, screening labs are performed and a search may be conducted to identify a suitably matched CB unit.

Study Products—Allogeneic Umbilical Cord Blood

Allogeneic unrelated donor CB units utilized for this trial will be obtained from the Carolinas Cord Blood Bank, an FDA licensed Public Cord Blood Bank at Duke University Medical Center. CB donors must be eligible for donation to a public cord blood bank for allogeneic use. Donor eligibility screening via questionnaires is performed in accordance with CFR 1271.75 and infectious disease testing is performed in accordance with CFR 1271.80 and 1271.85. The unit must also have an appropriate degree of HLA matching and meet product specifications as detailed below.

All potential study participants will undergo high resolution HLA typing at HLA-A, B, and HLA-DRB1 via blood or buccal swab. Patients receiving allogeneic CB will have HLA typing performed on two separate samples for confirmation. Allogeneic units that are potential matches will initially be identified from a search of the Carolinas Cord Blood Bank. The best available HLA-matched (≥4/6), using intermediate level matching at HLA Class I A and B and high resolution-allele level matching at HLA Class II, DRB1, CB unit with a pre-cryopreservation nucleated cell dose ≥12×10⁷ cells/kg will be selected. Once a unit is selected, HLA typing will be used to confirm the original HLA typing and to select the best matching unit. When possible, at least 1 match at each HLA loci will be prioritized. A CB unit must be at least 4/6 HLA-matched with the patient.

Recipients' ABO/Rh blood typing will be obtained. CB units will not be selected based on ABO typing. However, an Rh negative CB unit will be selected for Rh negative female participants to avoid Rh sensitization in young females.

Results of initial testing at the cord blood bank must include a pre-cryopreservation TNCC, viability and sterility culture. Pre-cryopreservation TNCC must be ≥12×10⁷ cells/kg to target administration of 10×10⁷ cells/kg post thaw, sterility cultures must have been negative, total viability must have been ≥85%, and CD34+ cell viability must have been ≥70%.

A test vial or segment must be available from each CB unit for potency testing and confirmatory HLA typing. The segment will be detached from the candidate unit and tested for potency and identity (HLA-confirmatory typing) per Standard Operating Procedures in the CCBB at Duke. Units will be deemed acceptable for the trial if viability of the CD45 cell population is ≥40% and viability of the CD34 cell population is ≥70%. CFU growth, expression of aldehydehydrogenase and CD34 will be described but will not be a specification for study enrollment.

Prior to the patients' arrival, their designated CB unit will be transferred from the Carolinas Cord Blood Bank to the Duke STCL, located in the same building, where it will be stored in a liquid nitrogen freezer until the day of infusion. On the infusion day, the CB will be thawed and washed in dextran/albumin and resuspended in an appropriate volume based on recipient weight for administration to the patient the standard fashion (Rubinstein et al. Proc Natl Acad Sci USA. 1995, 92(22):10119-10122) per SOP STCL-PROC-036. At the time of thawing, standard studies listed (see Table 1) will be performed. Only TNCC is utilized for release. A maximum dose of 10×10⁷ TNC/kg will be prepared for infusion in a syringe or bag and infused over 2-25 minutes.

TABLE 1
Post-Thaw Cord Blood Unit Testing

| Test | Specifications |
|---|---|
| Total Nucleated Cell Count (TNCC) | Report; used to calculate |
| | final dose |
| Viability | Report |
| Viability of the CD34+ population* | ≥70% |
| Viability of the CD45+ population | ≥40% |
| Sterility** | No Growth |
| Colony Forming Unit (CFU) growth | Report |
| ALDH^{br} as a percentage of CD45+ cells | Report |

*Viability of the CD34+ cells post-thaw was previously tested on a segment and required to meet the specification of ≥70%. Therefore, for the clinical product, we will report but not use the postthaw viability as a release criteria.
**If a positive culture is obtained after product administration, a plan is put into effect to notify the clinical and study teams and treat the patient if indicated.

Study Products—Human Umbilical Cord Tissue-Derived Mesenchymal Stromal Cells (hCT-MSC)

hCT-MSCs are manufactured under cGMP in a clean room ISO 7 facility and are a product of allogeneic cells manufactured from digested umbilical cord tissue that is expanded in culture, cryopreserved and banked. hCT-MSCs are manufactured in the Duke CT2 GMP cell manufacturing lab from umbilical cord tissue harvested from the placenta from normal term deliveries where the baby's cord blood was donated to the Carolinas Cord Blood Bank, an FDA-licensed, FACT-accredited, public cord blood bank at Duke University Medical Center, after written informed consent from the donor baby's mother. Cord tissue is harvested from the placentas of male babies delivered by elective C-section after a normal, full-term pregnancy. Donor screening questionnaires are completed by the maternal donor, and maternal blood is tested for communicable diseases by the CLIA-certified donor screening laboratory at the American Red Cross in Charlotte, N.C. Donors must be eligible for donation to a public cord blood bank for allogeneic use. After delivery of the placenta and cord, the cord blood is aseptically drained from the placenta. Then the cord is dried and cleaned with chloropreps, separated from the base of the placenta, placed in a sterile bottle containing Plasmalyte A, and transported to the Robertson Clinical and Translational Cell Therapy CT2 GMP cell processing laboratory at room temperature in a validated container.

In the clean room manufacturing suite, in a biosafety cabinet, the cord tissue is removed from the media, placed in sterile dishes, cut into small pieces and then minced and digested in the Miltenyi Biotec GentleMacs Octo Dissociator with GMP-grade enzymes: hyaluronidase, DNase, collagenase, papain. The resultant cell suspension is placed in culture in Prime XV MSC Expansion XSFM (Irvine Scientific) media with 1% platelet lysate and grown to confluence (˜7-14 days) to establish the P0 culture. To establish the master cell bank, P0 is harvested and cryopreserved in cryovials with Cryostor 10 media (BioLife), and stored in the vapor phase of liquid nitrogen. P1 and P2 cultures are grown under similar conditions, in HYPERFlasks or HYPERStacks without platelet lysate, as needed to create the working cell bank and product for administration, respectively. Cells from P1 and P2 are removed from plastic cultureware using TrypLE (Gibco). The final product is derived from the P2 cultures which are harvested into plasmalyte with 5% human serum albumin, washed and cryopreserved in compartment cryobags containing 50-100 million cells in a final concentration of 10% DMSO with dextran (Akron Scientific). On the day of administration, one compartment is thawed, diluted in 10-40 mLs of plasmalyte IV solution, placed in a syringe or bag and transported to the bedside for administration over 30-60 minutes.

At each passage, the cell product is characterized by assessing cell surface phenotype by flow cytometry and functional assays via T-cell proliferation and organotypic models of microglial activation. Each lot, prior to cryopreservation of P2, will also be tested for sterility, endotoxin and mycoplasma and these tests must meet specifications. For dosing, release testing after thaw and dilution will include TNCC and viability via cellometer. Patients will be dosed with 2×106 hCT-MSCs/kg based on the post thaw count.

Process and Final Formulation

hCT-MSC is manufactured from a single umbilical cord tissue in a series of three steps that generate a master cell bank, a working cell bank, and the study product. The product for each step is cryopreserved in a controlled rate freezer and stored in the vapor phase of liquid nitrogen. At P2, a representative cryobag is thawed and qualified prior to the treatment of any patients with that lot of product. Testing for product release includes total nucleated cell count, viability, phenotype, functional assays, endotoxin, mycoplasma, gram stain and sterility. Each lot of cells is also tested for adventitial viruses prior to cryopreservation.

On the day of treatment, cells are thawed per SOP STCLAOP-028 JA2 and then diluted in 10-40 mLs of plasmalyte A+5% human serum albumin (HSA). An aliquot is removed for cell count, viability, and sterility culture. If the cells are ≥70% viable, the final product volume is adjusted to deliver 2×10⁶ cells/kg to the study subject. The cells are delivered to the bedside in a syringe containing plasmalyte A, 5% HSA, and residual DMSO. Any removed cell suspension is inoculated into aerobic and anaerobic culture bottles for sterility testing. The cells have a four-hour expiry at room temperature post thaw.

The hCT-MSC final product will be released conditionally for administration to the patient after testing a post thaw cell count and viability. Final release will occur after the 14-day sterility culture period for the study product. In the event that a sterility culture turns positive after administration of the product, the organism will be identified and antibiotic sensitivities performed. The patient's family will be contacted to determine if they are symptomatic (i.e. fever or other signs of infection). Asymptomatic patients will be observed but will not be treated with antibiotics. Symptomatic patients will be evaluated and treated accordingly, with blood cultures and antibiotics as appropriate. All patients receiving a product with subsequent positive sterility test will be followed with daily contact by a study nurse for 14 days after the positive sterility test is noted.

Further manufacturing and testing details may be found in the U.S. Provisional Application Ser. 62/652,722, filed Apr. 4, 2018, the contents of which are hereby incorporated by reference in their entirety.

Study Products—Donor Screening for CB and hCT-MSC

Donor screening and testing is performed per Carolinas Cord Blood Bank standard operating procedures to meet all requirements in 21CFR Part 1271. The screening and testing is current with recommendations and is approved by the FDA under biological license number 1870. Maternal donors of umbilical cord blood are screened and tested for HIV-1, HIV-2, HIV-O, hepatitis B virus (HBV, surface antigen and core antibody), hepatitis C virus (HCV) antibody, Treponema pallidum (syphilis), Creutzfelds-Jakob Disease (CJD, screening only), Chagas Disease, human T-lymphotropic virus types 1 and 2 (HTLV-1, HTLV-2) and total antibodies against CMV. Nucleic acid testing for HIV-1/2/O, HBV, West Nile Virus and HCV are also performed on maternal blood. Screening for Zika virus may also performed.

Because the cord tissue used for this study will be obtained from donors consented for cord blood donation to the Carolinas Cord Blood Bank, they will undergo donor screening and infectious disease testing per Carolinas Cord Blood Bank standard operating procedures. The cord blood-associated maternal samples and cord tissue MSC samples will be retained as reference samples for future testing as part of this study.

Study Products—Packaging of Study Products

All cellular products receive a unique identification number (ISBT Demand 128 bar code) to ensure product integrity and maintain chain of custody. The clinical site or cord blood bank assigns an ISBT Demand 128 bar code label to the CB unit or hCT-MSC product, which is placed on the product bag/syringe directly or via tie tag. Products are transported from the STCL to the infusion site in a validated cooler by a trained courier.

Study Products—Administration of Study Product

Patients will arrive in clinic on the morning of their scheduled infusion. A peripheral IV will be placed either by an anesthesiologist, clinical staff or study staff and premedication with Benadryl 0.5 mg/kg/dose IV and Solumedrol 0.5-1 mg/kg IV will be administered. Allogeneic CB products will be administered intravenously over 5 to 25 minutes under direct physician supervision. hCT-MSC products will be administered intravenously over 30-60 minutes under direct supervision. Vital signs (heart rate, blood pressure, temperature, respiratory rate) will be checked upon arrival to the clinic and as clinically indicated. Pulse oximetry will be monitored continuously throughout the infusion and for at least 5 minutes post infusion. Patients will be hydrated with standard intravenous fluids as tolerated and observed for at least one hour post infusion.

Study Plan—Overview

Parents/Guardians who have previously contacted our program and have a child who may meet eligibility criteria for this study will be notified that this study is available. After initial contact, parents/guardians of potential research participants will have an initial phone interview with study personnel to describe the study, verify basic eligibility criteria, and confirm their interest in participation. The participant's eligibility will then be screened through review of medical records, video, laboratory testing, and imaging under a separate screening protocol.

Once all screening is complete and the patient is likely to meet study criteria, a suitable unrelated donor CB unit will be identified at the Carolinas Cord Blood Bank. The CB unit will be screened as described in section 6. Participants will then travel to Duke for their first visit. On day 1, written informed consent will be obtained. Patient eligibility will be confirmed by a physical observation and verification of cerebral palsy diagnosis and GMFCS level. If no exclusion criteria are realized, the participant will be randomized to a treatment arm. During their first visit, all participants will have physical therapy evaluations, and a subset of patients will undergo brain MRI. Participants will have study infusions as determined by their assigned treatment arm (at baseline only for AlloCB; at 12-months only for Natural History; at baseline, 3-, and 6-months for MSCs).

Participants will be evaluated the day after each infusion, and parents will be contacted for phone follow-up ˜2 weeks after each infusion. All participants will return to Duke six (motor assessments) and 12 months (motor assessments and brain MRI) after the baseline visit. Participants on the MSC arm will also return at three months for an hCT-MSC infusion. A remote safety assessment will be performed via phone or email at 24 months post-infusion.

Study Plan—Patient Screening

Initial patient screening will be conducted with informed consent under a separate protocol and will include a review of medical records, videos, and initial laboratory testing. If no exclusion criteria are identified, informed consent will be obtained over the phone, the patient will be randomized to treatment arm. If indicated (AlloCB and Natural History arms), an unrelated donor CB unit will be identified at the Carolinas Cord Blood Bank. Participants will travel to Duke for initial evaluation. Evaluations and treatments will be conducted in the outpatient setting. A physical exam and baseline GMFCS assessment will be conducted to confirm eligibility, and the participant undergo the remainder of the study evaluations.

Study Plan—CB Unit Selection

For participants randomized to the AlloCB and Natural History arms, an allogeneic unrelated donor CB unit will be identified at the Carolinas Cord Blood Bank. HLA typing will be obtained on the patient, and the best available HLA-matched CB unit with a precryopreservation nucleated cell dose ≥12×10⁷ cells/kg will be chosen. When possible, at least 1 match at each HLA loci will be prioritized. An Rh negative CB unit will be selected for Rh negative female participants to avoid Rh sensitization in young females.

Once a suitable allogeneic CB unit has been deemed an acceptable match, a sample of the CB unit will be tested for potency in the Duke STCL. If results of these tests are satisfactory, the CB unit will be delivered to the Duke STCL in the frozen state.

Study Plan—Study Product Infusion

On the day of infusion, CB cells or hCT-MSC product will be prepared by the STCL and provided for infusion of the patient in the outpatient clinic under the supervision of the study team and Pediatric Blood and Marrow Transplant Program staff. A peripheral IV will be placed by clinical staff, anesthesia or a member of the study team. Prior to the study infusion, premedications (Benadryl and Solumedrol) will be administered. CB cells will have a four-hour expiry at room temperature post-thaw.

Allo CB infusion will be given over approximately 5-25 minutes and hCT-MSC infusions over 30-60 minutes using standard practices. The child will receive 1-1.5× maintenance IV fluids as described below and be observed in the clinic for a minimum of one hour after the infusion. Patients will be discharged from clinic after at least one hour providing all vital signs are at their baseline and they are awake and asymptomatic with no evidence of toxicity. Patients will be evaluated by study staff the day after the infusion to assess for any infusion-related adverse reactions or complications. A phone call to parents/guardians by study staff to assess safety of the infusion will be conducted two weeks after the infusion.

Maintenance IV Fluid Rate (Holliday-Segar
Method from Harriet Lane Handbook)

| Body weight | mL/kg per day | |
|---|---|---|
| 1st 10 kg | 100 | divided by 24 hr/day |
| 2nd 10 kg | 50 | divided by 24 hr/day |
| each add'l kg | 20 | divided by 24 hr/day |

If a patient has evidence of illness on the day of planned infusion, including but not limited to fever >38.5° C., vomiting, diarrhea, or respiratory distress, the infusion will be postponed.

Study Plan—Care During Unexpected Events

In the event that a patient develops signs or symptoms of anaphylaxis including urticaria, difficulty breathing, cough, wheezing, or vomiting during their CB infusion, the infusion will be terminated and appropriate medical therapy initiated.

Study Plan—Motor Assessments

Gross Motor Function Measurement-66 (GMFM-66): The GMFM-66 is a standardized observational instrument designed and validated to measure change in gross motor function over time in children with cerebral palsy. Developmental curves of expected progression have been published for children ages 2-12 years (Hanna et al. Phys Ther. 2008, 88(5):596-607; Rosenbaum et al. Jama. 2002, 288(11):1357-1363) allowing for the calculation of future expected scores based on the baseline age, GMFCS level, and GMFM-66 score. The GMFM-66 consists of 66 items, divided into five categories: lying and rolling, sitting, crawling and kneeling, standing, and walking, running, and jumping.

Each item is scored on a four-point Likert scale. The GMFM-66 is a subset of the GMFM-88, which contains an additional 22 items, primarily in the lying and rolling category. Both measures have been validated in children with cerebral palsy from 5 months to 16 years of age. A 5-year old child without motor disabilities is able to reach the maximum score (Russell et al. Gross Motor Function Measure (GMFM-66 GMFM-88) User Manual. London: Mac Keith Press; 2013). A computer program, the Gross Motor Ability Estimator; is used to calculate the GMFM-66 total scores. The primary endpoint of this study is the difference between a child's actual and expected changes in GMFM-66 score 12 months after the initial study infusion. Control (placebo) and treated patients will be compared.

When possible, the entire GMFM-88 will be performed, and subsets may be analyzed as exploratory endpoints.

Peabody Developmental Motor Scales (PDMS-2): The PDMS-II is a standardized assessment of early childhood motor development that evaluates both gross and fine motor skills. It is designed for children from birth through 5 years of age. The assessment is composed of six subtests that measure interrelated motor abilities that develop early in life (i.e., reflexes, stationary, locomotion, object manipulation, grasping, and visual-motor integration). Gross Motor Quotient, Fine Motor Quotient, and Total Motor Quotient composite scores are obtained. For this study, the Gross Motor Quotient will be obtained and analyzed as a secondary endpoint.

Study Plan—Functional and Quality of Life Assessments

Pediatric Evaluation of Disability Inventory-Computer Adaptive Test (PEDI-CAT): The PEDI-CAT measures abilities in three functional domains: Daily Activities, Mobility, and Social/Cognitive. The computerized adaptive version is intended to provide an accurate and precise assessment of a child's abilities while increasing efficiency and reducing respondent burden by utilizing item response theory statistical models to determine which items are assessed within each domain based on responses to prior items.

Pediatric Quality of Life Inventory 4.0, Generic Core Scale and Cerebral Palsy Module (PedsQL) (Varni et al. Developmental medicine and child neurology. 2006, 48(6):442-449). The PedsQL General Core Scales and Cerebral Palsy Module are composed of parallel child self-report and parent proxy-report formats. The 35-item PedsQL Cerebral Palsy Module encompasses seven scales and generates a standard score: (1) Daily Activities (9 items), (2) School Activities (4 items), (3) Movement and Balance (5 items), (4) Pain and Hurt (4 items), (5) Fatigue (4 items), (6) Eating Activities (5 items), and (7) Speech and Communication (4 items).

Study Plan—Imaging Assessments

Participants' brain imaging obtained previously as standard of care will be reviewed by a member of the Brain Imaging Analysis Center (BIAC) team to determine if accurate anatomical image parcellation would be likely on a brain MRI. Those participants for whom usable data is likely to be obtained (estimated as approximately two-thirds of eligible participants) will undergo brain MRI with diffusion tensor imaging (DTI). Diffusion weighted images will be acquired on a 3 Tesla GE scanner (Waukesha, Wis.). T1-weighted images will be obtained with an inversion-prepared 3D fast spoiled-gradientrecalled (FSPGR) pulse sequence. These images will be analyzed to obtain measures of whole brain connectivity.

Statistical Considerations—Study Design

This study is a phase I/II, prospective, randomized, open-label trial designed to provide interval estimates of the 12-month change in motor function after treatment with AlloCB and hCT-MSC, provide additional data to the clinical trials community on the natural history of the motor function in CP over short-term (less than 1 year) time periods relevant to conduct of clinical trials, and assess the safety of repeated doses of hCTMSC and a single dose of AlloCB in children with cerebral palsy.

Children ages 2-5 years with cerebral palsy due to hypoxic ischemic encephalopathy, stroke, or periventricular leukomalacia will be eligible to participate. All participants will ultimately be treated with an allogeneic cell product at some point during the study. Participants will be randomized (1:1:1) to one of three arms: (1) the “AlloCB” arm will receive one allogeneic CB infusion at the baseline visit; (2) the “MSC” arm will receive three hCT-MSC infusions, one each at baseline, three months, and six months; the “natural history” arm will not receive an infusion at baseline but will receive an allogeneic CB infusion at 12 months. The occurrence of adverse events will be evaluated at 3, 6, 12, and 24 months post-randomization in all participants. Motor function outcome measures will be assessed at baseline, six-months, and one-year time points in all participants. Duration of study participation will be 24 months from the time of the baseline visit. Randomization will be stratified by age (2-3 years vs. 4-5 years) and GMFCS Level (I/II or III/IV).

Statistical Considerations—Accrual

It is estimated that up to 8-12 research participants will be enrolled each month and that approximately 12-15 months of accrual will be necessary to enroll 90 participants.

Statistical Considerations—Study Duration

Each subject's participation in the study will be 24 months, with clinic visits occurring during the first 12 months and a remote safety assessment at 24 months. Given that accrual will take up to 15 months it is estimated that the remote safety assessment will be conducted on that last patient 39 months (3.25 years) after the study opens.

Statistical Considerations—Primary and Secondary Endpoints

The primary endpoint of this study is the difference between a child's observed and expected changes in GMFM-66 score 12 months after the initial study infusion. This study will provide separate interval estimates of the mean of this outcome measure in patients assigned to the hC-MSC, AlloCB, and Natural History arms at 12-months. The secondary endpoint of this study is the number of adverse events occurring over a 12-month period post-treatment with hCT-MSC or AlloCB.

Statistical Considerations—Sample Size and Power Calculations

The sample size for this study was selected to provide a high level of precision for estimating the mean of the observed minus expected 12-month change on the GMFM-66 in each of the study arms, and to provide a high probability of detecting commonly occurring adverse events after infusion with AlloCB or hCT-MSC.

As shown in Table 2 below, a sample size of 30 patients per group provides a 95.8% probability of detecting common adverse events that occur in 10% of infusions (with hCT-MSC or AlloCB). This sample size also provides a 78.5% probability of observing events that occur in 5% of infusions, and a 26.0% probability of observing rare events that occur in 1% of infusions.

TABLE 2
Probability of Observing One or More
Events with Various Sample Sizes*

| | Probability (%)* | | | |
|---|---|---|---|---|
| True Probability of an Event (%) | N = 20 | N = 30 | N = 40 | N = 50 |
| 1 | 18.25 | 26.0 | 33.1 | 39.5 |
| 5 | 64.2 | 78.5 | 87.1 | 92.3 |
| 10 | 87.8 | 95.8 | 98.5 | 99.5 |
| 20 | 98.8 | 99.9 | 100.0 | 100.0 |
| 50 | 100.0 | 100.0 | 100.0 | 100.0 |

*Binomial probability of 1 or more independent events.

The sample size for this study must also support estimation of the mean observed minus-expected GMFM-66 change score at 12 months post-intervention with MSC, AUCB, and in the Natural History arm. Thus, three interval estimates will be constructed using the t-distribution as follows.

$$( \overline{x} - t α / 2 * \frac{s}{\sqrt{n}} , \overline{x} + t α / 2 * \frac{s}{\sqrt{n}} )$$

The margin of error E is the confidence interval half-width:

$$E = t α / 2 * \frac{s}{\sqrt{n}}$$

The margin of error for this study was selected as 2 points with a confidence level of 95%. The following formula was solved iteratively to obtain the sample size for each treatment group.

$$N = {( \frac{t α / 2 * s}{E} )}^{2}$$

The standard deviation, s, was estimated using 36 participants in the CP-AC trial who met age and GMFCS inclusion criteria for the present study: 5.16 (95% CI: 4.18, 6.13). Starting with a sample size of 20, and assuming a standard deviation of 5.16, a total of 3 iterations were required to reach a final group sample size of 28 as shown in Table 3 below.

TABLE 3

| | | Degrees of | | |
|---|---|---|---|---|
| Iteration # | Starting N | Freedom | tα_{/2} | Ending N |
| 1 | 20 | 19 | 2.093 | 29 |
| 2 | 29 | 28 | 2.048 | 28 |
| 3 | 28 | 27 | 2.052 | 27 |

Therefore, a group size of 28 patients allows for 95% confidence in the estimation of the mean 12-month observed-minus-expected GMFM-66 change score in one of the study arms (Natural History, MSC or AUCB) with a margin of error of no more than 2. This sample size is also concordant with what is required (N=30) for reasonable probability of detecting commonly occurring adverse events, as described above. Finally, if the standard deviation of the secondary outcome measure is as high as that indicated by the upper limit of the 95% confidence interval from the CP-AC study (6.13 points) then a sample of 126 patients allows for a margin of error no larger than −2.5 points for each of the three interval estimates.

The total sample size for this study is therefore set at 90 patients (30 per group).

Statistical Considerations—Analysis Plan

Analysis Populations: The following populations are defined to support analyses of the primary and secondary endpoints.

Intention to Treat Population
This population will include all enrolled and randomized participants according to their assigned treatment. The primary endpoint will be evaluated in this population.

Safety Population
The safety population defines the patients in whom the secondary endpoint will be evaluated and will include all subjects who received at least 1 infusion. Analyses of the Safety Population will be conducted using an as-treated approach, which considers each patient according the treatment actually received rather than the treatment they were assigned.

Timing of Analyses

The analysis of the primary and secondary outcome measures will be conducted when the last patient reaches their 12-month visit. An update will be made to the safety analyses when the last patient reaches their 24-month visit.

Demographics, Baseline Characteristics, and Disposition

Demographics and baseline characteristics will be summarized for all research participants and separately by randomized assignment. Characteristics to be examined include age, sex, race/ethnicity, baseline GMFM-66 score, GMFCS level, and etiology of CP. The number of participants entering and completing the study will be diagrammed using the CONSORT guidelines.

Analysis of the Primary and Secondary Endpoints

The occurrence of adverse events in the Safety Population will be summarized descriptively in tables and figures for all subjects and separately by treatment received. Estimates of the mean observed-minus-expected GMFM-66 change score at 12 months will be reported in the Intention to Treat Population along with 95% confidence intervals as described above.

### Example 2: Phase I Study of hCT-MSC, an Umbilical Cord-Derived Mesenchymal Stromal Cell Product, in Newborn Infants with Moderate or Severe Hypoxic-Ischemic Neonatal Encephalopathy

Purpose

The purpose of this Phase 1 study is to assess the safety of one and two intravenous infusions of human umbilical cord tissue-derived mesenchymal stromal cells (hCT-MSC), the first administered in the first 48 postnatal hours, and the second at two months postnatal age, in term and near term infants with moderate to severe neonatal hypoxic-ischemic encephalopathy (HIE). The first three enrolled infants will get only the first, early dose. The second three enrolled infants will get both doses.

Study Rationale and Hypotheses

The mechanistic rationale and overarching theory of this line of investigation is that hCT-MSCs can act through paracrine and allocrine mechanisms to modulate on-going inflammation and/or immune pathology in the brain and possibly protect neurons from further damage. The hypothesis of this phase I clinical trial is that administration of hCT-MSCs in one or two doses of cells will be safe in newborn infants born at 36 weeks gestation or later, who suffer from moderate to severe hypoxic-ischemic neonatal encephalopathy.

In many contexts, MSCs dampen, rather than augment, immunological and inflammatory responses. Documented mechanisms include shifts in effector T cells such as generation of regulatory T cell populations and changes in monocyte/dendritic cell cytokine generation leading to anti-inflammatory cytokines. Therefore, it is plausible to consider a population of MSCs as an immunological and/or anti-inflammatory agent. Animal models of neonatal HIE have revealed evidence of increased microglial activation. In addition, neutrophils accumulate in the central nervous system vasculature. Myeloid cells, T cells and natural killer cells infiltrate injured areas of the brain during the recovery phase, suggesting that immune and/or inflammatory mediated brain damage plays a role in the etiology of ASD as discussed above (Hagberg 2015). Thus, hCT-MSCs may be a candidate therapy for HIE because of the immunomodulatory activities of MSCs. Additionally, a multiple dosing regimen may improve the overall rate and duration of response.

Study Objectives

To determine the safety of single and repeated intravenous doses of hCT-MSC in neonates with HIE.

Risks and Benefits

The potential risks associated with infusion of hCT-MSC include a reaction to the product (rash, shortness of breath, wheezing, difficulty breathing, hypotension, swelling around the mouth, throat or eyes, tachycardia, diaphoresis), transmission of infection, and HLA sensitization. Theoretical risks that must be considered but have not been associated with MSC administration in humans include the possibility of immune suppression and ectopic tissue formation. Cord blood collected with the donor cord tissue used to manufacture hCT-MSC is screened for infection, and the product must meet release criteria prior to infusion (described below). Other than a single dose of hydrocortisone before the hCT-MSC dose in the first postnatal days, or methylprednisolone and diphenhydramine prior to the 2nd infusion of hCT-MSC, participants will not receive immunosuppressive therapy prior to or after infusion of hCT-MSC cells.

Potential benefits of this intervention include the possibility that hCT-MSC may, via direct or indirect mechanisms, induce changes that result in the reduction of the participant's HIE-related pathologies and improvement in abilities affected by hypoxic-ischemic injury, in particular, motor and/or cognitive function.

Study Design—General Design

This study is a phase I, prospective, open-label trial designed to assess the safety of one or two intravenous doses of hCT-MSC in newborn infants with moderate to severe HIE who are recipients of therapeutic hypothermia. Children born at 36 0/7 weeks gestation or later who have moderate to severe hypoxic-ischemic encephalopathy and are receiving therapeutic hypothermia will be eligible to participate. All participants will receive intravenous infusion(s) of hCT-MSCs. The first cohort of three patients will receive a single dose in the first 48 postnatal hours. If there are no safety concerns, the second cohort of three patients will receive two doses, with the first dose given in the first 48 postnatal hours and the second dose given approximately two months after the first dose. All participants will be receiving hypothermia for moderate to severe neonatal encephalopathy, as indicated by long-standing Duke Intensive Care Nursery criteria, based on the criteria used in the Eunice Kennedy Shriver NICHD Neonatal Research Network's Optimizing Hypothermia multisite clinical trial (Shankaran et al., JAMA 2014, 312(24):2629-2639). The main endpoint is safety, for which acute infusion reactions and incidence of infections will be assessed. HIE-specific outcome measures, described below, will be assessed at baseline and six months and one year from baseline and results will be described.

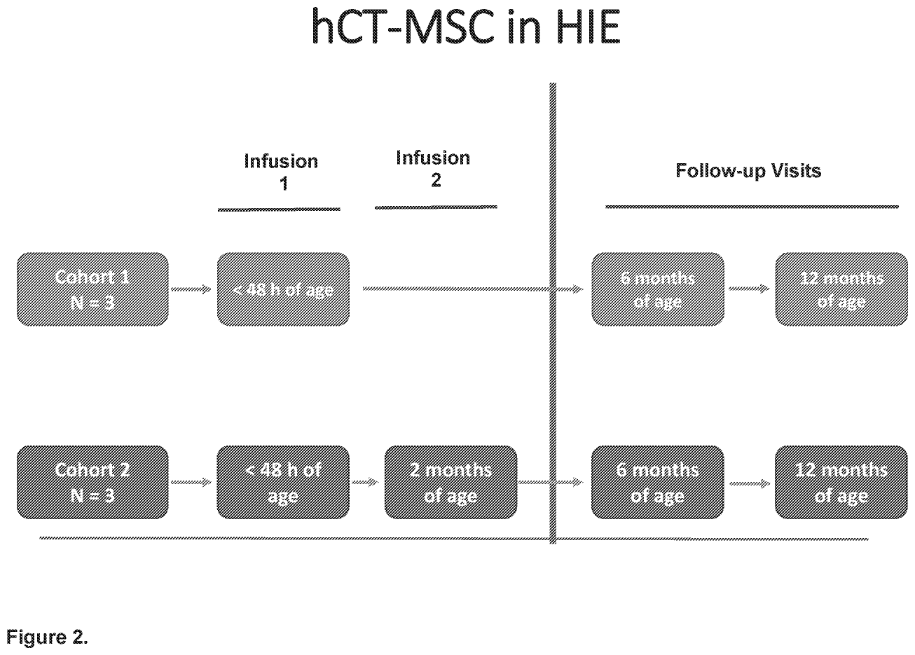

A study flow chart is provided in FIG. 2.

Study Design—Study Endpoints

The primary endpoint of this study is safety, which will include assessing the incidence of acute infusion reactions and infections.

Additionally, HIE- and HIE-related complications will be assessed to describe any changes in condition after product administration. Death or moderate-severe impairment will be collected as secondary outcomes. This will be determined with Bayley III scores in all three domains. We will report all the scores for all enrolled infants, and also report how many babies survived with all three Bayley III domain scores greater than or equal to 85. NICU outcomes will also be considered secondary outcomes including mortality, seizures, pulmonary hypertension, need for nitric oxide and need for ECMO, need for G-tube feeding at discharge, and discharge on anti-epileptic medications.

Primary Safety Endpoints: will be assessed by:
1 Incidence of infusion reactions: for this study, infusion reactions are defined as anaphylactic or anaphylactoid reactions with clinical signs inclusive of skin rashes, bronchospasm, angioedema, myocardial infarcts, arrhythmias, and acute lung injury
2. Incidence of infections: for this study, infections recorded as safety endpoints will be defined as bacterial, viral or fungal infections identified by culture or molecular methodologies within two weeks after administration of hCT-MSC.

Secondary/Exploratory Endpoints:

In addition to safety measures, the following HIE-specific endpoints will be assessed:
1 Death prior to discharge from initial hospitalization
2. Need for anti-epileptic medications at discharge home
3. Need for g-tube or other non-oral feedings at discharge home
4. Pulmonary hypertension (defined by clinicians' use of inhaled nitric oxide initiated after hCT-MSC infusion)
5. Need for extra-corporeal membrane oxygenation (ECMO) for any reason, after hCT-MSC infusion.
6. 1 year (12-16 postnatal months) Bayley III assessments in cognitive, language and motor development.

Research Participant Selection and Withdrawal—Study Population

Six newborn infants with moderate to severe HIE, receiving therapeutic hypothermia for HIE.

Research Participant Selection and Withdrawal—Inclusion Criteria
1. 36 0/7^{th} weeks gestation or older at the time of delivery.
2. Able to receive one dose of hCT-MSCs in the first 48 postnatal hours, and for the second cohort of 3 infants, be available for the second infusion of cells two months after the first infusion (some infants may be outpatients at this point).
3. Willingness to return for one year assessments.
4. Signs of encephalopathy within 6 hours of age utilizing the two step (A and B) approach used in the Network's Optimizing Hypothermia Study, with clinician decision to initiate therapeutic hypothermia for moderate or severe hypoxic-ischemic encephalopathy as determined by the exam used clinically to determine eligibility for therapeutic hypothermia (Shankaran et al., JAMA 2014, 312(24):2629-2639). Exams at Duke that are used to determine use of hypothermia are done by NNPs or MDs who have been trained and certified in the Network's Optimizing Cooling trial, or are discussed and reviewed by a trained examiner. The exams are documented using a ‘smart phrase’ in the Duke Electronic Health Record.

The eligibility criteria for therapeutic hypothermia in detail:
Infants will be evaluated in two steps; evaluation by clinical and biochemical criteria (Step A, which is either A1 or A2, depending on available information and severity of blood gas abnormalities), followed by a neurological exam (Step B)
Once infant meets either A1 or A2, proceed to neurologic examination. (See part B)
   The presence of moderate/severe encephalopathy (a “2” or a “3”) defined as seizures OR presence of signs in 3 of 6 categories in the table below. For the categories with more than one item, such as PRIMITIVE REFLEXES, the item (SUCK, MORO) with the highest score determines the level of encephalopathy assigned for that category
   The neurologic examination will be performed by a physician examiner, or a non-physician examiner who has reviewed the qualifying exam with the clinician deciding on use of therapeutic hypothermia.

Steps A1 and A2. All infants will be evaluated for the following:
1. Is the baby ≥36 weeks gestation?
2. Is there a history of an acute perinatal event (abruptio placenta, cord prolapse, severe FHR abnormality: variable or late decelerations)?
3. Is the Apgar score <5 at 10 minutes or is there a continued need for ventilation initiated at birth and continued for at least 10 minutes?
4. What is the cord pH or first postnatal blood gas pH at <1 hour?
5. What is the base deficit on cord gas or first postnatal blood gas at <1 hour?

If infant meets criteria A1 or A2 (see Tables 4 and 5 below) and criteria B, and does not meet exclusion criteria, the infant is eligible and is therefore eligible for study enrollment.

TABLE 4

| | IF BLOOD GAS IS NOT AVAILABLE |
|---|---|
| | OR pH between 7.0 and 7.15, |
| IF BLOOD GAS IS | OR |
| AVAILABLE: | BASE DEFICIT 10 to 15.9 mEq/L |
| A1 | A2 |
| Answer to #1 is ‘YES’ AND | Answer to #1 is ‘Yes’ AND |
| Cord pH or first postnatal blood | Acute perinatal event (#2) and either |
| gas within 1 hour with pH <7.0 | An Apgar score <5 at 10 minutes (#3) |
| (#4) | |
| OR | OR |
| Base deficit on cord gas or first | Continued need for ventilation initiated |
| postnatal blood gas within 1 | at birth and continued for at least 10 |
| hour at >16 mEq/L (#5) | minutes (#3) |

TABLE 5
Part B: Neurologic Assessment

| | SIGNS OF HIE IN EACH LEVEL | | |
|---|---|---|---|
| | Normal/ | MODERATE | |
| CATEGORY | Mild HIE | HIE | SEVERE HIE |
| 1. LEVEL OF | 1 | 2 = Lethargic | 3 = Stupor/coma |
| CONSCIOUSNESS | | | |
| 2. SPONTANEOUS | 1 | 2 = Decreased | 3 = No activity |
| ACTIVITY | | activity | |
| 3. POSTURE | 1 | 2 = Distal flexion, | 3 = Decerebrate |
| | | complete extension | |
| 4. TONE | 1 | 2a = Hypotonia | 3a = Flaccid |
| | | (focal or general) | 3b = Rigid |
| | | 2b = Hypertonia | |

5. PRIMITIVE REFLEXES

| | | | |
|---|---|---|---|
| Suck | 1 | 2 = Weak or has | 3 = Absent |
| | | bite | |
| Moro | 1 | 2 = Incomplete | 3 = Absent |

6. AUTONOMIC SYSTEM

| | | | |
|---|---|---|---|
| Pupils | 1 | 2 = Constricted | 3 = Deviation/ |
| | | | dilated/non- |
| | | | reactive to light |
| Heart rate | 1 | 2 = Bradycardia | 3 = Variable HR |
| Respiration | 1 | 2 = Periodic | 3 = Apnea or |
| | | breathing | requires ventilator |
| | | | 3a = on vent with |
| | | | spont breaths |
| | | | 3b = on vent |
| | | | without spont |
| | | | breaths |

Research Participant Selection and Withdrawal—Exclusion Criteria
1. Major congenital or chromosomal abnormalities
2. Severe growth restriction (birth weight <1800 g)
3. Opinion by attending neonatologist that the study may interfere with clinical treatment or safety of subject
4. Moribund neonates for whom no further treatment is planned
5. Infants whose mothers have unknown serologies for Hepatitis B or HIV
6. Infants born to mothers are known to be HIV, Hepatitis B, Hepatitis C or who have active syphilis or CMV infection in pregnancy
7. Infants suspected of overwhelming sepsis
8. ECMO initiated or likely in the first 48 hours of life
9. ALL blood gases (cord and postnatal) done within the first 60 minutes had a pH >7.15 AND base deficit <10 mEq/L (source can be arterial, venous or capillary)
10. Mother with documented Zika infection during this pregnancy
11. Availability of autologous cord blood collected and usable in the randomized trial of autologous volume- and red blood cell-reduced cord blood cells (clinicaltrials.gov identifier NCT02612155)

Acknowledging that other conditions may develop, and congenital conditions (inherited or acquired) may become apparent before the second infusion, the following exclusion criteria are added to the 11 criteria listed above and are applied to the second infusion.

1. Infectious:
a. Known active CNS infection
b. Evidence of uncontrolled infection based on records or clinical assessment
c. Known HIV positivity

2. Medical:
a. Known metabolic disorder
b. Known abnormal thyroid function (patients with treated hypothyroidism with a normal TSH may be included)
c. Known mitochondrial dysfunction
d. History of unstable epilepsy or uncontrolled seizure disorder, infantile spasms, Lennox Gastaut syndrome, Dravet syndrome, or other similar chronic seizure disorder
e. Active malignancy or prior malignancy that was treated with chemotherapy
f. History of a primary immunodeficiency disorder
g. History of autoimmune cytopenias (i.e., ITP, AIHA)
h. Coexisting medical condition that would place the child at increased risk for complications of study procedures
i. Concurrent genetic or acquired disease or comorbidity(ies) that could require a future stem cell transplant
j. Known significant sensory (e.g., blindness, deafness, uncorrected hearing impairment) or motor impairment
k. Impaired renal or liver function as determined by serum creatinine >1.5 mg/dL or total bilirubin >1.3 mg/dL, except in patients with known Gilbert's disease
l. Significant hematologic abnormalities defined as: Hemoglobin <10.0 g/dL, WBC <3,000 cells/mL, ALC <1000/uL, Platelets <150×10e9/uL
m. Evidence of clinically relevant physical dysmorphology indicative of a genetic syndrome as assessed by the PIs or other investigators, including a medical geneticist and psychiatrists trained in identifying dysmorphic features associated with neurodevelopmental conditions.

3. Current/Prior Therapy:
a. History of prior cell therapy
b. Current or prior use of IVIG or other anti-inflammatory medications with the exception of NSAIDs
c. Current or prior immunosuppressive therapy
d. No systemic steroid therapy that has lasted >2 weeks; topical and inhaled steroids are permitted.

Research Participant Selection and Withdrawal—Research Participant Recruitment and Screening

We will screen all infants admitted to the Duke Intensive Care Nursery who are >35 6/7th weeks gestation, for whom a decision was made by the clinical team to offer therapeutic hypothermia for moderate to severe neonatal hypoxic-ischemic encephalopathy, and cooling was or will be initiated in the first 6 postnatal hours.

If cord blood was not collected for the currently enrolling randomized trial of autologous cord blood cells for neonatal HIE (see exclusion criteria above), members of the clinical caregiving team will introduce the basic concepts of this phase I study of allogeneic hCT-MSCs to the eligible newborn infant's family. If the family is interested in learning more, the members of the clinical team will contact the research team. A member of the research team will then connect with the potentially eligible infant's family to discuss the study at length, and discuss consent for the infant's participation in the study.

Research Participant Selection and Withdrawal—Early Withdrawal of Research Participants

Criteria for Removal from Protocol Therapy:
1. Diagnosis of a genetic or infectious disease while under evaluation or on study.
2. Change in medical condition that precludes study participation.

Patients who are off protocol therapy are to be followed until they meet off-study criteria (see below). Follow-up data will be obtained on off-protocol participants unless consent is withdrawn. Subjects taken off study prior to the first or, if in the second cohort, the second infusion of hCT-MSC will be considered not evaluable and can be replaced with another subject.

Off-Study Criteria:
1. Death.
2. Lost to follow-up.
3. Withdrawal of consent for any further data collection.
4. Completion of the final study visit.

Study Product—Human Umbilical Cord Tissue-Derived Mesenchymal Stromal Cells (hCT-MSC)

hCT-MSCs are a product of allogeneic cells manufactured from digested umbilical cord tissue that is expanded in culture, cryopreserved and banked. hCT-MSCs are manufactured from umbilical cord tissue donated to the Carolinas Cord Blood Bank, an FDA-licensed, FACT-accredited, public cord blood bank at Duke University Medical Center, after written informed consent from the baby's mother. Cord tissue is harvested from the placentas of male babies delivered by elective C-section after a normal, full-term pregnancy. Donor screening questionnaires are completed by the maternal donor, and maternal blood is tested for communicable diseases by the CLIA-certified donor screening laboratory at the American Red Cross in Charlotte, N.C. Donors must be eligible for donation to a public cord blood bank for allogeneic use. After delivery of the placenta and cord, the cord blood is aseptically drained from the placenta. Then the cord is dried and cleaned with chloropreps, separated from the base of the placenta, placed in a sterile bottle containing Plasmalyte A, and transported to the Marcus Center for Cellular Cures (MC3) GMP cell processing laboratory at room temperature in a validated container.

In the clean room manufacturing suite, in a biosafety cabinet, the cord tissue is removed from the media, placed in sterile dishes, cut into small pieces and then minced and digested in the Miltenyi Biotec GentleMacs Octo Dissociator with GMP-grade enzymes: hyaluronidase, DNase, collagenase, papain. The resultant cell suspension is placed in culture in Prime XV MSC Expansion XSFM (Irvine Scientific) media with 1% platelet lysate and grown to confluence (˜7-14 days) to establish the P0 culture. To establish the master cell bank, P0 is harvested and cryopreserved in cryovials with Cryostor 10 media (BioLife), and stored in the vapor phase of liquid nitrogen. P1 and P2 cultures are grown under similar conditions, in hyperflasks or hyperstacks without platelet lysate, as needed to create the working cell bank and product for administration, respectively. Cells from P1 and P2 are removed from plastic cultureware using TrypLE (Gibco). The final product is derived from the P2 cultures which are harvested into plasmalyte with 5% human serum albumin, washed and cryopreserved in 5 compartment cryobags (Syngen) in 5 mL containing 50-100 million cells in a final concentration of 10% DMSO with dextran (Akron Scientific). On the day of administration, one compartment is thawed, diluted in 6-9 mls of plasmalyte-A+5% HSA IV solution, placed in a syringe or bag and transported to the bedside for administration over 30-60 minutes.

At each passage, the cell product is characterized by assessing cell surface phenotype by flow cytometry and functional assays via T-cell proliferation and organotypic models of microglial activation. Each lot, prior to cryopreservation of P2, will also be tested for sterility, endotoxin and mycoplasma and these tests must meet specifications. For dosing, release testing after thaw and dilution will include TNCC, viability via cellometer, gram stain and endotoxin. Participants will be dosed with 2×10⁶ hCT-MSCs/kg based on the post thaw count.

One lot of hCT-MSCs will be selected for this clinical trial. The lot will be tested in 1-2 patients at each dose level, per Table 6 below. A total of 6 participants will be treated with 2 dosing regimens. For the three participants who are planned to receive two doses, each dose will consist of 2×10⁶ hCT-MSCs/kg, and doses will be given two months apart.

TABLE 6

| | | | hCT-MSC |
|---|---|---|---|
| | Patient # | # of Doses | Lot # |
| Cohort 1 | 1 | 1 | 1 |
| | 2 | 1 | 1 |
| | 3 | 1 | 1 |
| Cohort 2 | 4 | 2 | 1 |
| | 5 | 2 | 1 |
| | 6 | 2 | 1 |

Study Product—Donor Screening and Testing

Because the cord tissue used for this study will be obtained from donors consented for cord blood donation to the Carolinas Cord Blood Bank, they will undergo donor screening and infectious disease testing per Carolinas Cord Blood Bank standard operating procedures. The cord blood-associated maternal samples and cord tissue MSC samples will be retained as reference samples for future testing as part of this study.

Donor screening and testing is performed per Carolinas Cord Blood Bank standard operating procedures to meet all requirements in 21CFR Part 1271. The screening and testing is current with recommendations and is approved by the FDA under biological license number 1870. Maternal donors of umbilical cord blood are screened and tested for HIV-1, HIV-2, hepatitis B virus (HBV, surface and core antigen), hepatitis C virus (HCV), Treponema pallidum (syphilis), CJD (screening only), Chagas, human T-lymphotropic virus types 1 and 2 (HTLV-1, HTLV-2) and CMV. Nucleic acid testing for HIV-1/2/O, HBV, WNV and HCV are also performed on maternal blood. Screening and testing of maternal donors for Zika virus is also performed.

Study Product—Process and Final Formulation

hCT-MSC is manufactured from a single umbilical cord tissue in a series of three steps that generate a master cell bank, a working cell bank, and the study product. The product for each step is frozen and stored in vapor phase in liquid nitrogen freezer. At P2, a representative cryobag will be thawed and qualified prior to the infusion in study participants with that lot of product. Testing will include cell count, viability, phenotype, functional assays, endotoxin, mycoplasma, gram stain and sterility.

On the day of infusion, cells are thawed per SOP CT2-MSC-006, diluted in 10-40 mls of plasmalyte-A+5% HSA, and an aliquot removed for cell count, viability, and sterility culture. If the cells are ≥70% viable, the final product volume is adjusted to deliver 2×10⁶ cells/kg to the study subject. The cells are delivered to the bedside in a bag or syringe containing plasmalyte-A, 5% HSA, and residual DMSO. Any removed cell suspension is inoculated into aerobic and anaerobic culture bottles for sterility testing. The cells have a four-hour expiry post thaw.

The hCT-MSC final product will be released conditionally for administration to the patient after testing a post thaw cell count and viability. Final release will occur after the 14-day sterility culture period for the study product. In the event that a sterility culture turns positive after administration of the product, the organism will be identified and antibiotic sensitivities performed. Clinicians caring for the infants in the study will be informed of the culture results by study staff. For the 2nd infusion, which for some infants could occur in an outpatient setting, the patient's family will be contacted to determine if they are symptomatic (for example, have fever). Clinicians providing primary care for the subjects will assess need for clinical evaluation and treatment. All patients receiving a product with subsequent positive sterility test will be followed with daily contact by a study team member for 14 days after the positive sterility test is noted.

Study Products—Packaging of Study Products

All umbilical cord tissues will be assigned an ISBT Demand 128 bar code label or unique identifier, which is carried through to all in-process and final hCT-MSC products. In addition, the MC3 GMP facility will provide a final product label for each hCT-MSC product. The product label will include a space to affix the bar code label as well as space for the subject number, date and time of product expiry, and any other pertinent information. As a subject is enrolled, a subject number will be assigned which will link to the 12 digit ISBT number bar code number assigned to the umbilical cord blood tissue. The final product will be assigned a lot number (manufacturing operation number) and expiry date and time that will be denoted on the Certificate of Analysis and product label. The subject number and ISBT bar code number of the product will be also listed on the Certificate of Analysis. All products will be transported from the GMP laboratory of the Marcus Center for Cellular Cures to the Intensive care nursery (for the first dose) or to the Valvano Day Hospital (2nd dose if baby is to be dosed as an outpatient) in a validated cooler by courier.

Study Product—Recipient's Mother's Screening

Because babies enrolled in the study will be receiving a cell product, regulations require that the mothers of cell-recipient babies be screened as if their child was donating to the the cord blood bank. All testing described for the umbilical cord tissue donor mothers is also required for mothers of enrolled infants (blood samples for HIV-1, HIV-2, hepatitis B virus (HBV, surface and core antigen), hepatitis C virus (HCV), Treponema pallidum (syphilis), CJD (screening only), Chagas, human T-lymphotropic virus types 1 and 2 (HTLV-1, HTLV-2) and CMV. Nucleic acid testing for HIV-1/2/O, HBV, WNV and HCV and Zika virus). Mothers of enrolled infants must also respond and complete the health questionnaire completed by mothers providing permission for their baby's cord blood or cord tissue to be collected, processed and stored and/or used allogeneically. Obtained samples will be retained as reference samples for future testing as part of this study.

Study Products—Administration of Study Product

For babies meeting entry criteria, the first dose of cells will be infused intravenously as soon as possible, with the target being in the first 48 postnatal hours, during therapeutic hypothermia. For the first infusion, infants will be pretreated with hydrocortisone, 1 mg/kg IV 30-60 minutes prior to each infusion if the subject was not on hydrocortisone for clinical purposes. Vital signs (heart rate, blood pressure, temperature, respiratory rate) will be monitored in the intensive care nursery as clinically indicated. Pulse oximetry will be monitored continuously throughout the infusion and for at least 60 minutes post infusion. Subjects will be observed and vital signs recorded every 15 minutes post infusion for the first hour, and then documented per standard of care for the next four hours

For the second infusion, some study subjects may have been discharged home. These patients will be admitted to the infusion center on the day of their scheduled infusion. Patients may require some sedation prior to the IV placement if they are unable to remain still or cooperate. A peripheral IV will be placed by clinical or study staff. Patients will be premedicated with diphenhydramine 0.5 mg/kg/dose IV and methylprednisolone 0.5-1 mg/kg IV, per standard procedures for post-neonatal cell infusions. The hCT-MSCs will be administered intravenously over 30-60 minutes. Vital signs (heart rate, blood pressure, temperature, respiratory rate) will be monitored upon arrival to the clinic and monitored as clinically indicated. Pulse oximetry will be monitored continuously throughout the infusion and for at least 5 minutes post infusion. Patients will be observed for at least one hour post infusion.

Study Products—Safety Follow-Up

On Day 1 following each infusion, the participant will be seen by study staff to assess for any infusion related adverse reactions or complications. For those subjects receiving the 2nd infusion, the study staff will follow up with the parent or guardian via phone or email 1 day following the infusion. At 14 days post each administration of hCT-MSC, a member of the study team will contact the clinical staff (if the patient is still admitted in the intensive care nursery) as well as the parent or guardian via phone or email to assess patient status and any adverse events. A questionnaire will be administered at 2 weeks, and 2, 6 and 12 months after the initial dose to assess for serious adverse events.

Study Plan—Overview

Parents/Guardians who have a newborn infant meeting inclusion criteria will be notified by clinical staff caring for their infant that this study is available. After initial contact, parents/guardians of potential research participants will have an initial phone or in person interview with study personnel to describe the study, verify basic eligibility criteria, and confirm their interest in participation.

Once all screening is complete and the patient is likely to meet study criteria, the study will be introduced to the family by the clinical team. The study team will attempt to obtain informed consent by the research staff if the parents have expressed interest in the study to the clinical team. If the child is deemed eligible and the parent(s) agree, he/she will be enrolled on study and scheduled to receive hCT-MSC infusion(s). Participants will be evaluated the day after each infusion either in person or by phone call and bedside caregivers and/or parents will be contacted 14 days after each infusion for follow up safety evaluations. The second three participants, if discharged from the hospital, will return to Duke for scheduled hCT-MSC infusion and monitoring 2 months after the infusion of cells in the first 48 postnatal hours. All participants will return to Duke's Special Infant Care Clinic for follow-up assessments at six months and one year following their initial dose for repeated neurodevelopmental evaluations and safety follow-up.

Study Plan—Participant Screening

A waiver of HIPAA Authorization and informed consent will be requested to allow study staff to screen the Duke Intensive Care Nursery admissions for infants meeting inclusion criteria and not meeting exclusion criteria. If no exclusion criteria are identified, the study staff will contact the clinicians caring for the potential subject to discuss clinical trial eligibility. A patient must be approved by both the study team and the clinical team to proceed with study enrollment. Should a concern for a previously undiagnosed condition or genetic finding arise during the screening process, this will be discussed with the patient's parent(s)/guardian(s) and a referral will be made to an appropriate medical or psychiatric provider for evaluation and treatment, if indicated.

Study Plan—hCT-MSC Infusion

All subjects will receive at least one infusion of allogeneic hCT-MSC cells. On the day of infusion, hCT-MSC cells will be thawed and prepared by the MC3 GMP laboratory per standard operating procedure and provided for infusion of the patient in the Duke Intensive Care Nursery, or the clinic under the supervision of the study team and Pediatric Blood and Marrow Transplant Program staff. Baseline vital signs (heart rate, blood pressure, temperature, respiratory rate) will be obtained. If an IV is not available to use for infusion of the hCT-MSCs, a peripheral IV will be placed by clinical staff, anesthesia or a member of the study team.

Prior to the infusion of cells, premedications (hydrocortisone for the first 48 postnatal hour infusion, and diphenhydramine and methylprednisolone for the two month infusion) will be administered. The hCT-MSCs will be infused over 30-60 minutes. The child will be observed in the intensive care nursery, or the clinic for a minimum of 1 hour after the infusion. IV fluids (D5 ½ NS) at 1.5 maintenance will be provided.

Patients receiving the two month dose will be discharged from clinic after at least 1 hour providing all vital signs are at their baseline and they are asymptomatic with no evidence of toxicity. Patients will be evaluated by study staff the day after the infusion to assess for any infusion-related adverse reactions or complications. A phone call to parents/guardians by study staff to assess safety of the infusion will be conducted 14 days after the infusion.

For the two month infusion, if a patient has evidence of illness on the day of planned infusion, including but not limited to fever >38.5° C., vomiting, diarrhea, or respiratory distress, the infusion will be postponed.

Study Plan—Care During Unexpected Events

In the event that a patient develops signs or symptoms of anaphylaxis including urticaria, difficulty breathing or worsening respiratory status (increase in respiratory support, an absolute increase in fiO2 of more than 10% during the infusion; need to initiate inhaled nitric oxide during the infusion), cough, wheezing, or vomiting during his/her hCT-MSC infusion, the infusion will be terminated and appropriate medical therapy initiated.

Study Plan—Baseline Laboratory Testing

The following baseline laboratory assessments will be performed: Maternal Baseline
Maternal blood for ARC Donor panel testing
Maternal ARC Donor medical history questionnaire

All Participants' Baseline Prior to dose administered in the first 48 postnatal hours
Participant's HLA typing (by buccal swab)
CBC with differential (part of SOC)
Chemistry panel, including bilirubin (part of SOC)
Type and Screen (part of SOC)

Participants' Cohort 2—Second Infusion; labs drawn before second infusion at 2 months of age.
CBC with differential
Complete Metabolic Panel
Panel Reactive Antibody

Monitoring During and after Infusions—Vital Signs

Vital signs will be assessed pre-infusion, for 60 minutes post infusion and per hospital routine thereafter.

Monitoring During and After Infusions—Metabolic Status

Daily chemistries are routinely obtained in infants with moderate to severe HIE when they are treated with hypothermia. Serum electrolytes, CBC, BUN, and creatinine, and frequently liver function tests are monitored at baseline, and then daily. We will record results of metabolic laboratories collected for clinical purposes during the cooling period and the first 24 hours post re-warming in the case report forms.

Monitoring During and after Infusions—Respiratory Status

A daily blood gas is standard in infants with moderate to severe HIE. Results from daily blood gases obtained for clinical purposes during cooling and for the first 24 hours after re-warming will be collected in the case report forms.

Monitoring During and after Infusions—Neurologic Status

A neurological assessment will be performed at baseline, daily during cooling, and at discharge. This will be performed by a trained examiner.

Monitoring During and after Infusions—Hematologic Status

Monitoring of coagulation studies is considered routine for infants with HIE. PT/PTT results obtained for clinical purposes during cooling and for the first 24 hours after re-warming will be recorded in the case report forms.

Monitoring During and after Infusions—Neuroimaging

An MRI is routinely obtained on HIE infants. Results from the standard of care MRI will recorded for the study, and we will record and report results in terms of injury scores developed by the NICHD Neonatal Research Network (NRN) to be extracted from the clinical interpretations of images (Shankaran et al., Archives of Disease in Childhood, Fetal and Neonatal Edition 2012, 97(6):F398-404).

Monitoring During and after Infusions—Post-Infusion Assessments

Post-Infusion Assessments:
24-hour post infusion assessment
14 days post each infusion: (phone or in person) questionnaire.
2 months of age: (phone or in person) questionnaire.
6 months of age: In person assessment, including laboratory studies on samples obtained from all participants (CBC w/diff, CMP, Direct and indirect Coombs, PRA) and questionnaire.
12-16 months of age: In person assessment, including Bayley III exam, and labs (CBC w/diff, CMP, Direct and indirect Coombs, PRA) and questionnaire.

Statistical Considerations—Study Design

This study is a phase I, prospective, open-label trial designed to assess the safety of one, and two intravenous doses of hCT-MSC in newborn infants with moderate to severe neonatal HIE. Newborn infants with moderate to severe neonatal HIE, who are treated with therapeutic hypothermia, may be eligible to participate. All participants will receive an intravenous infusion(s) of hCT-MSC. The first cohort of three patients will receive a single dose. If there are no safety concerns, the second cohort of three patients will receive a second dose, given at approximately two months of age. The main endpoint is safety, for which acute infusion reactions and incidence of infections will be assessed from data collected during the hospitalization, and from the phone and in-person surveys. Vital signs, metabolic, respiratory, neurologic and hematologic outcomes, described above, will be assessed. Neurodevelopmental outcomes at twelve (12-16) months after the initial hCT-MSC infusion, and results will be described.

Statistical Considerations—Accrual

It is estimated that approximately one research participant will be enrolled each month and that approximately 6-8 months of accrual will be necessary to enroll 6 subjects. To ensure that potential study treatment-related risks to participants are minimized, an interval of at least one month will be observed after the final dose in one cohort before the final dose is given to the first subject in the subsequent cohort. For example, there will be at least one month between the time that subject #3 (last subject in cohort 1) receives the hCT-MSC infusion and the time that subject #4 (first subject in cohort 2) receives the second hCT-MSC infusion.

Statistical Considerations—Study Duration

Research participants will be followed for safety for 10-12 months after the administration of their final dose of hCT-MSC. There will be follow-up questionnaires at 14 days after each infusion and at 2 months to assess safety outcomes and overall progress if any routine visits are missed or re-scheduled. There are in person assessments at 6 and 12 (12-16 months) which are the times for usual clinical visits with the special infant care clinic to assess progress.

Statistical Considerations—Demographics and Baseline Characteristics

Demographics and baseline characteristics will be summarized for all research participants. Maternal characteristics will be recorded, including maternal age, race gravida/parity status. Complications of pregnancy to record include chronic hypertension, type 1 or type 2 diabetes, hyper- or hypothyroidism, preeclampsia, and antepartum hemorrhage.). Maternal medications (chronic and intrapartum) will be recorded. Maternal delivery complications (fetal heart rate decelerations, cord prolapse, uterine rupture, placental abruption, maternal pyrexia, shoulder dystocia, maternal hemorrhage,) will be recorded. Study subjects' characteristics to be examined include gestational age, sex, race/ethnicity, and baseline level of encephalopathy (moderate or severe, based on the NICHD study criteria), inborn vs. outborn status, 1, 5, 10, 15 and 20 minute Apgar scores, qualifying blood gas and neurologic assessment details. Details of resuscitation will also be recorded (need for positive pressure and mechanical ventilation, need medications during resuscitation, cooling and cell infusions). Medication and ventilation needs during cooling, with specific respiratory support and medication use at the time of first infusion, will be recorded.

Statistical Considerations—Primary Endpoint

The primary safety measure will be the incidence of infusion reactions and infections post-infusion. This will be assessed at the time of infusion, 24 hours after each infusion, 14 days after each infusion, upon any return evaluation by the clinical team, and at six months after each of the 6 subjects' final infusion (the first 3 will have only one infusion, so infections and infusion reactions will be collected and reported for 12 months after this single infusion, with the second recording at the 12-16 month neurodevelopmental assessment visit. For the second three infants who receive a second infusion at 2 months, infections and infusion reactions will be collected and reported until the participant is 12-16 months old. Results for the primary and secondary outcomes will be reported descriptively.

Statistical Considerations—Sample Size and Power Calculations

Given the study design, the sample size selected is based on clinical judgment, and not on statistical considerations.

Statistical Considerations—Secondary Endpoints

Secondary efficacy endpoints include survival and neurodevelopmental assessments twelve months (12-16 months of age) after the first/final dose, age at discharge, neurological exam at discharge, need for anti-epileptics at hospital discharge, non-oral feeding at hospital discharge, pulmonary hypertension confirmed by echocardiogram and defined by need for inhaled nitric oxide, and need for ECMO.

For neurodevelopmental assessments, infants will have Bayley III neurodevelopmental assessments results in 3 domains: motor, cognitive, and language development.

Exploratory endpoints will be reported using summary tables, figures, and data listings. The results will be summarized using descriptive statistics and statistical testing as appropriate. Continuous secondary endpoints will be summarized using mean, standard deviation, CV %, median, minimum, and maximum. Summaries of changes from baseline to include 95% confidence intervals and p-values associated with paired t-test will also be provided. If data are not normally distributed, we will use a transformation to approximate a normal distribution or use a non-parametric test. Changes from baseline will be assessed for vital signs before, during and after cell infusions.

Categorical exploratory endpoints will be summarized by presenting the number (frequency) and percentage in each category. Categorical data will be presented as frequencies and percentages. Shift tables for changes from baseline of categorical outcomes may be produced, whenever appropriate. Characteristics to be assessed for changes over time include whether or not infants who were not mechanically ventilated prior to cells required mechanical ventilation, infants not receiving nitric oxide received nitric oxide, infants not on anti-seizure medications were subsequently diagnosed with seizures and were treated with anti-seizure medications.

All statistical tests will use an alpha level of 0.05 in order to declare significance. For secondary efficacy outcomes, there is no pre-specified hierarchical order for assessment, and no adjustments of the significance level for multiple testing will be performed.

We claim:

1. A method of treating a patient with cerebral palsy comprising administering a therapeutically effective amount of human allogeneic umbilical cord-derived mesenchymal stromal cells (hCT-MSCs) to the patient.

2. The method of claim 1, wherein the hCT-MSCs are administered systemically.

3. The method of claim 2, wherein the hCT-MSCs are administered intravenously.

4. The method of claim 1, wherein the patient is administered hCT-MSCs three times in a six month period.

5. The method of claim 4, wherein the patient is administered hCT-MSCs at baseline, at three months, and at six months.

6. The method of claim 1, wherein the patient is administered hCT-MSCs at a dose of at least about 2×10⁶/kg.

7. A method of treating a patient with hypoxic-ischemic encephalopathy (HIE) comprising administering a therapeutically effective amount of human allogeneic umbilical cord-derived mesenchymal stromal cells (hCT-MSCs) to the patient.

8. The method of claim 7, wherein the hCT-MSCs are administered systemically.

9. The method of claim 8, wherein the hCT-MSCs are administered intravenously.

10. The method of claim 7, wherein the patient is administered hCT-MSCs three times in a six month period.

11. The method of claim 10, wherein the patient is administered hCT-MSCs at baseline, at three months, and at six months.

12. The method of claim 7, wherein the patient is administered hCT-MSCs at a dose of at least about 2×10⁶/kg.

13. The method of claim 7, wherein the patient with HIE is a newborn 36 weeks gestation or later, who suffers from moderate to severe hypoxic-ischemic neonatal encephalopathy.

14. The method of claim 13, wherein the patient is administered hCT-MSCs in a single dose in the first 48 postnatal hours.

15. The method of claim 14, wherein the patient is administered a second dose of hCT-MSCs approximately two months after the first dose.

16. The method of claim 13, wherein the hCT-MSCs are administered systemically.

17. The method of claim 16, wherein the hCT-MSCs are administered intravenously.

18. The method of claim 13, wherein the patient is administered hCT-MSCs at a dose of at least about 2×10⁶/kg.
